(19) United States
(12) Patent Application Publication
(10) Pub. No.: US 2024/0303492 A1
(43) Pub. Date: Sep. 12, 2024

(54) GENERATING NEURAL NETWORK MODELS, CLASSIFYING PHYSIOLOGICAL DATA, AND CLASSIFYING PATIENTS INTO CLINICAL CLASSIFICATIONS

(71) Applicant: Oxford University Innovation Limited, Oxford (GB)

(72) Inventors: Yanting SHEN, Oxford (GB); Robert CLARKE, Oxford (Headington) (GB); Tingting ZHU, Oxford (GB); David CLIFTON, Oxford (GB)

(21) Appl. No.: 18/280,751

(22) PCT Filed: Mar. 4, 2022
(86) PCT No.: PCT/GB2022/050573

§ 371 Date: Sep. 7, 2023

(30) Foreign Application Priority Data
Mar. 11, 2021 (GB) 2103370.9

(51) Int. Cl. G06N 3/082; G06N 3/0464; G16H 10/60

(52) U.S. Cl. CPC G06N 3/082; G06N 3/0464; G16H 10/60

(57) ABSTRACT

Computer-implemented methods for generating neural networks, and methods for classifying physiological data and patients based on the generated networks, are provided. The methods comprise determining values of a plurality of hyperparameters based on one or more properties of the received input data, which may be physiological data. A neural network comprising a plurality of layers is generated based on the hyperparameters, and is trained using the input data. If a first predetermined condition is not met, the values of one or more of the hyperparameters are updated. The steps of generating and training a neural network are repeated until the first predetermined condition is met. When the first predetermined condition is met, one of the trained neural networks is selected and is output.

### INTRODUCTION

The invention relates to methods for generating neural networks, in particular to automatic neural network design for particular applications, such as classification of physiological data and classification of patients into clinical classifications.

Neural networks are being applied in increasingly many fields for a variety of purposes, including classification of data. One popular type of neural network is the convolutional neural network. Convolutional neural networks (CNN) are networks with at least one layer of convolutional operation, and are an example of a weight sharing mechanism.

The first CNN, LeNet-5, was proposed by [13] to read handwritten digits. LeNet-5 started using repeating structures comprised of one or more convolutional layers, followed by a pooling layer. These repeating structures were then followed by a flatten layer to concatenate the last output tensor into one long vector, then connect to several densely connected layers for classification. LeNet-5 also popularised the heuristic of reducing fₕ and f_{w} and increasing f_{c} as the layers go deeper. The convolution-pooling blocks served as feature extraction layers, and the fully-connected layers, typically having a decreasing number of neurons, reduced dimensions gradually, and the final layer served as the classifier.

AlexNet was proposed by Alexander Krizhevsky [12] and won ILSVRC 2012 [16], which has a profound impact on deep learning history as it convinced the computer vision community of the power of deep learning. AlexNet has a similar architecture as LeNet-5 but is a much larger network, with 8 layers and over 62 million parameters. K. Simonyan and A. Zisserman [18] took the “principled” hyperparameter selection to another level to build VGG-16. They used an increasing number of neurons as the layers go deeper, resulting in a total of 16 layers and 138 million parameters. The relatively rational choice of hyperparameters makes it attractive to the developers. VGG-16 won ILSVRC in 2014. The development of the state-of-the-art CNNs has the trend of increasing depth, but the number of parameters does not necessarily increase.

Before a neural network can be trained on a particular data set, design choices must be made about the architecture of the neural network, for example the number and dimension of the layers of the network. The current state-of-the-art method for this stage of neural network development is trial and error. A designer will choose the architecture, test it, and make changes based on their own experience and intuition about what will improve performance. Some general principles may be followed, for example using a small model when the training data is scarce, and a large model when the training data is abundant. However, it is rare for the neural network architecture to be designed in any consistent and systematic way, for example based on the exact number of training examples.

The randomness inherent in neural network training due to random weight initialisation, stochastic gradient estimation, and other sources of randomness makes model development especially challenging. It can be unclear if the change in the performance is due to an intervention to change the architecture (such as adding layers and changing hyperparameters) or due to the randomness in training. Commonly, researchers train the model on the same set of hyperparameters several times before concluding the helpfulness or the harmfulness of an intervention. This is undesirable when the model becomes very large, and training once would take days to months.

In view of these limitations, it would be desirable to provide improved techniques for designing neural network architecture that are more consistent and which require less human input.

### Claim Counterparts

According to an aspect of the invention, there is provided a computer-implemented method for generating a neural network comprising: receiving input data; determining values of a plurality of hyperparameters based on one or more properties of the input data; generating, based on the values of the hyperparameters, a neural network comprising a plurality of layers; training the neural network using the input data and, at least if a first predetermined condition is not met, updating the values of one or more of the hyperparameters; repeating the steps of generating a neural network, and training the neural network until the first predetermined condition is met; selecting one of the trained neural networks; and outputting the selected neural network.

By choosing initial parameters of the neural network based on properties of the input data, and using an iterative method to develop the neural network, the method can consistently generate an architecture suitable for the input data for which the neural network is to be used.

In some embodiments, the plurality of layers comprises one or more pooling layers and one or more convolutional layers between each pooling layer, the plurality of hyperparameters comprising the number of pooling layers and the number of convolutional layers between each pooling layer.

Convolutional neural networks (CNN) are an example of a weight sharing mechanism. CNNs allow reuse of “feature detectors” at multiple locations in the input data. For example, in an image processing application, the CNN should be able to detect eyes anywhere in the image. CNNs also share weights within the same layer in order to reduce the number of parameters, effectively reducing overfitting and lowering computational cost.

In some embodiments, the pooling layers are maxpooling layers.

Maxpooling layers provide a simple mechanism for reducing dimensionality that reduces computational cost.

In some embodiments, the input data is periodic time series data, and in the step of determining values of the plurality of hyperparameters, the number of pooling layers is determined based on a number of samples in the time series data per period of the time series data.

By basing the number of pooling layers on the timescale of periodic data, overfitting can be minimised by preventing fitting across larger timescales that would be inappropriate based on the periodicity of the data.

In some embodiments, the number of pooling layers is determined according to:

nₘₐₓₚₒₒₗ=┌logₚ(fₛτ)┐

where nₘₐₓₚₒₒₗ is the number of pooling layers, p is a predetermined parameter quantifying a reduction in dimensionality by each pooling layer, τ is a predetermined estimate of the period of the time series data, and fₛ is a sampling frequency of the time series data.

This particular form of the dependence ensures an appropriate number of pooling layers based on the periodicity and the chosen degree of pooling at each pooling layer.

In some embodiments, the input data is non-periodic time series data, and in the step of determining values of the plurality of hyperparameters, the number of pooling layers is determined based on a number of samples in the time series data.

Where data is non-periodic, it is appropriate to fit across the full length of the input data.

In some embodiments, the number of pooling layers is determined according to:

nₘₐₓₚₒₒₗ=┌logₚ(D)┐

where nₘₐₓₚₒₒₗ is the number of pooling layers, p is a predetermined parameter quantifying a reduction in dimensionality by each pooling layer, and D is a number of samples of the time series data.

This particular form of the dependence ensures an appropriate number of pooling layers based on the length of the input data and the chosen degree of pooling at each pooling layer.

In some embodiments, the plurality of layers further comprises an activation layer following each convolutional layer.

Using activation layers standardises the output from the convolutional layers, giving more predictable training performance and reducing erroneous parameter choices during training.

In some embodiments, the activation layer comprises a rectified linear unit or a leaky rectified linear unit.

Rectified linear units or leaky rectified linear units are well-understood activation functions that ensure the output of convolutional layers will be (unstrictly) monotonic.

This improves training performance.

In some embodiments, updating the values of one or more of the hyperparameters comprises increasing the number of convolutional layers between each pooling layer.

Gradually increasing the number of convolutional layers allows the neural network depth to grow to an appropriate level to fit the input data. This reduces the likelihood of the neural network having too many layers (leading to overfitting, increased training time, and increased computational requirements) or too few layers (leading to reduced accuracy and performance).

In some embodiments, the input data is labelled input data and the neural network is trained using supervised learning.

Supervised learning is most appropriate for classification tasks, for example classification of physiological data.

In some embodiments, the plurality of layers comprises one or more pooling layers and one or more convolutional layers between each pooling layer, the plurality of hyperparameters comprising the number of pooling layers and the number of convolutional layers between each pooling layer; each convolutional layer has an associated plurality of parameters, and training the neural network comprises: choosing values of the parameters of the convolutional layers based on the values of the hyperparameters and the previous values of the parameters of the convolutional layers; calculating a training value of a loss function using an output of the neural network; and repeating the steps of choosing values of the parameters and calculating the value of the loss function until a change in the training value of the loss function over two or more consecutive steps of calculating the training value of the loss function is below a predetermined threshold.

Iterative training of the network allows the network to choose parameters appropriate for the input data.

In some embodiments, the training value of the loss function comprises a training loss calculated by evaluating the loss function on the output of the neural network applied to the input data.

Using a training loss value allows the supervised learning to iteratively improve its performance on the input data.

In some embodiments, the first predetermined condition is met when a validation value of a loss function of the neural network following the step of training the neural network is not lower than the validation value of the loss function of the neural network following the training of the previous neural network.

Using a validation loss value to evaluate performance of the architecture and choose when to change the architecture of the neural network provides independence between the training of the individual networks and the evaluation of their performance relative to one another.

In some embodiments, the method further comprises, after the first predetermined condition is met: generating, based on the values of the hyperparameters, a neural network comprising one or more skip connections between non-consecutive layers of the neural network; training the neural network comprising one or more skip connections using the input data and, at least if a second predetermined condition is not met, updating the values of one or more of the hyperparameters; and repeating the steps of generating a neural network comprising one or more skip connections and training the neural network comprising one or more skip connections until the second predetermined condition is met.

Adding skip connections can help to prevent vanishing gradient problems in neural network training, which cause stagnation of improvement between training iterations. However, skip connections can lead to the neural network to converge at a relatively shallow architecture, as the skip connections usually lead to a marked improvement in both training and validation losses. Therefore, it is advantageous to only add the skip connections at a later stage of the development of the architecture, once no further improvement is obtained from adding additional convolutional layers alone.

In some embodiments, the second predetermined condition is met when a validation value of a loss function of the neural network comprising one or more skip connections following the step of training the neural network comprising one or more skip connections is not lower than the validation value of the loss function of the neural network comprising one or more skip connections following the training of the previous neural network comprising one or more skip connections.

Using a validation loss value to evaluate performance of the architecture provides independence between the training of the individual networks and the evaluation of their performance relative to one another.

In some embodiments, the method further comprises, after the second predetermined condition is met: generating, based on the values of the hyperparameters, a neural network comprising one or more batch normalisation layers; training the neural network comprising one or more batch normalisation layers using the input data and, at least if a third predetermined condition is not met, updating the values of one or more of the hyperparameters; and repeating the steps of generating a neural network comprising one or more batch normalisation layers and training the neural network comprising one or more batch normalisation layers until the third predetermined condition is met.

Batch normalisation improves the stability of neural networks, and so is desirable in deep neural networks. It is also advantageous to add batch normalisation at a later stage of the architecture development, similarly as for skip connections, as this improves stability in earlier stages of the architecture development.

In some embodiments, the plurality of layers comprises a plurality of convolutional layers and an activation layer following each convolutional layer, and the neural network comprising one or more batch normalisation layers comprises a batch normalisation layer following each activation layer.

Including batch normalisation layers after each activation layer ensures that the input into each convolutional layer is normalised.

In some embodiments, the third predetermined condition is met when a validation value of a loss function of the neural network comprising one or more batch normalisation layers following the step of training the neural network comprising one or more batch normalisation layers is not lower than the validation value of the loss function of the neural network comprising one or more batch normalisation layers following the previous step of training the neural network comprising one or more batch normalisation layers.

As noted above, using a validation loss value to evaluate performance of the architecture provides independence between the training of the individual networks and the evaluation of their performance relative to one another.

In some embodiments, the validation value of the loss function comprises a validation loss calculated by evaluating the loss function on the output of the neural network applied to a validation data set.

Using a separate validation data set for the calculation of the validation loss ensures that the neural network is generalizable to data other than that used to train the neural network.

In some embodiments, the input data comprises time series data.

Neural networks of the type generated by this method are particularly suited to the analysis of time series data.

In some embodiments, the time series data is cyclic physiological data.

It is desirable to apply machine learning techniques to cyclic physiological data to aid in the classification of patient data and the identification of potential abnormalities.

In some embodiments, the time series data is electrocardiogram data.

Electrocardiogram (ECG) data is an example of physiological data which can classified in this manner by the neural networks generated using the present method.

In some embodiments, selecting one of the trained neural networks comprises selecting the trained neural network having a lowest validation value of a loss function.

Selecting the best-performing network based on validation loss is a straightforward way to provide an output of the method, which minimises any additional steps to provide the output and minimises computational cost.

In some embodiments, selecting one of the trained neural network comprises: training the neural network having a lowest validation value of a loss function a plurality of times to obtain a corresponding plurality of trained instances of the neural network having the lowest validation value of the loss function; and providing as the selected neural network an average ensemble of the trained instances.

Outputting an average ensemble of trained instances of the best-performing network can reduce variation due to the randomness of training. This can provide more consistent output of a better-performing neural network.

In some embodiments, the validation value of the loss function comprises a validation loss calculated by evaluating the loss function on the output of the trained neural network applied to a validation data set.

As noted above, using a separate validation data set for the calculation of the validation loss ensures that the neural network is generalizable to data other than that used to train the neural network.

In some embodiments, outputting the selected neural network comprises outputting the values of the hyperparameters used in generating the selected neural network.

The hyperparameters define the architecture of the neural network, so one desirable output is the architecture determined to be appropriate for a particular class of input data. The hyperparameters can then be used to generate neural networks with the optimal architecture for training on other data sets of the same type.

In some embodiments, the plurality of layers comprises one or more convolutional layers, each convolutional layer having an associated plurality of parameters, and outputting the selected neural network comprises outputting the values of the parameters of the convolutional layers.

It may also be desirable to output a fully-trained neural network, including the values of the parameters, depending on the application for which the neural network is to be used.

In some embodiments, the neural network further comprises a classification layer.

A classification layer can be used to classify input data into one of a plurality of classes, for example so that decisions can be based on the determination that a particular input data instances corresponds to a certain class.

In some embodiments, the times series data is physiological data, and the classification layer is configured to classify the input data into one of a plurality of clinical categories.

A particularly desirable application is to aid medical personnel in the diagnosis of clinical data by classifying the input into clinical categories.

According to another aspect of the invention, there is provided a method of classifying physiological data from a patient, the method comprising: receiving the physiological data; generating a neural network according to embodiments of the first aspect in which the time series data is physiological data and the network comprises a classification layer, and using the neural network to classify the physiological data (e.g. into one of a plurality of clinical categories).

The method of generating a neural network ensures that the neural network has an architecture that optimises performance and accuracy. Therefore, using neural networks generated using the method provides improvements in performance and accuracy when applied to the classification of physiological data from a patient.

According to a further aspect of the invention, there is provided a method of classifying a patient into a clinical category, the method comprising: receiving the physiological data; generating a neural network according to the embodiments of the first aspect in which the times series data is physiological data, and the classification layer is configured to classify the input data into one of a plurality of clinical categories; using the neural network to classify the physiological data; and classifying the patient into one of a plurality of clinical categories based on the classification of the physiological data from the classification layer of the neural network.

The invention may also be embodied in a computer program, computer-readable medium, or an apparatus.

### LIST OF FIGURES

Embodiments of the invention will now be described, by way of example only, with reference to the accompanying drawings in which corresponding reference symbols represent corresponding parts, and in which:

FIG. 1 is a flowchart of the method of generating a neural network;

FIG. 2 is a diagram of an exemplary baseline neural network;

FIG. 3 is a flowchart showing the steps in training a neural network;

FIG. 4 shows detail of the structure of a section of a neural network generated by an embodiment of the method of generating a neural network;

FIG. 5 is a flowchart of a method for classifying physiological data using a neural network generated using the method of generating a neural network;

FIG. 6 shows the split between training, validation, and test data for the data sets used to test the neural networks generated by the method of generating a neural network;

FIG. 7 shows the structure of the neural network generated based on the ICBEB data set;

FIG. 8 shows the structure of the neural network generated based on the PhysioNet data set; and

FIG. 9 shows the structure of the neural network generated based on the CKB data set.

### DETAILED DESCRIPTION

The present disclosure provides a computer-implemented method for generating a neural network. The method allows the automatic generation of neural networks (also referred to as models) based on the characteristics of input data in the form of a training data set to determine a network architecture best suited to the input data. The method may be referred to as “AutoNet” or the “AutoNet algorithm”.

The deep learning research community has long been searching for the “one-network-to-rule-them-all”. While the present disclosure does not attempt to build the “one-model-to-rule-them-all”, it customises neural networks for each application and input data set automatically, and uses a unified algorithm to determine the hyperparameters of the neural network.

The primary neural network architecture design consideration, after deciding on the model family (e.g. feed-forward, recurrent, or convolutional neural networks), is the width and depth of the network. The width refers to the number of neurons in each layer of the network, and the depth refers to how many layers the network contains. There is no consensus as to how to count the layers. Some authors count only one of the output and input layers, while others count both. Some authors only count layers with learnable parameters, while others also count layers without learnable parameters, such as pooling layers. Some authors count convolutional layers and activation layers separately, while others consider the convolutional and activation a single layer and call it a convolutional layer.

At present, the depth and width of a neural network is mostly designed by trial and error. The method disclosed herein allows these parameters, amongst others, to be determined automatically, based on principles of information theory. The depth of the network is determined using principles of reinforcement learning, and by adapting the model size according to training and validation losses. Each training example in the input data is regarded as one piece of information. The goal of the method is to create a neural network (also referred to as a “model”) that makes the best use of the training data set while also facilitating optimisation. As will be discussed and demonstrated below, this allows the network architecture to be determined in a more systematic and consistent way. In turn, this reduces the time needed to optimise the architecture, as well as providing better performing neural networks with lower memory requirements.

In the disclosure below, the method is demonstrated in application to generating a particular class of neural networks known as deep Layer-Wise Convex Networks (LCNs), which is a novel deep learning architecture family. However, the algorithm is also applicable to the generation of other types of neural network, and is not limited to the specific class of LCNs.

### Basic Formulation

Let us use supervised-class classification as an example, and denote the design matrix with X∈ [symbol] ^{D×m}, where D is the dimension of the feature vector, m is the number of training examples, Y∈ [symbol] ^{K×m} represents the one-hot-encoded training targets, where K is the number of classes. Let Ŷ represent the prediction of Y given by an L-layer neural network, then each layer of the network computes:

$${Z}^{[ l ]} = {W}^{[ l ]} {A}^{[ l - 1 ]} + {b}^{[ l ]} \qquad ( 1 )$$

$${A}^{[ l ]} = {g}^{[ l ]} ( {Z}^{[ l ]} ) \qquad ( 2 )$$

for l=0, 1, . . . , L. Layer o and layer L represent the input and the output layers, respectively; in other words, A^{[0]}=X, and A^{[L]}=Ŷ. A^{[l]}∈ [symbol] ⁿ^{^{[l]}}^{×m} is called the activation or output of layer l, g^{[l]} is (usually) the non-linear activation function of layer l; Z^{[l]}∈ [symbol] ⁿ^{^{[l]}}^{×m} is the affine transformation of the activations of layer l−1; W^{[l]}∈ [symbol] ⁿ^{^{[l]}}^{×n}^{^{[l-1]}} is the weight matrix pointing from layer l−1 to layer l in the forward pass; n^{[l-1]} and n^{[l]} are the number of neurons in layer l−1 and layer l, respectively. b^{[l]}∈ [symbol] ⁿ^{^{[l]}} is the bias vector of layer l.

### Layer-Wise Convex Networks

The LCN theory is derived from the assumption that the neural network comprises activation functions that are strictly monotonic. The LCN theory can be extended to non-strictly monotonic activation functions such as rectified linear unit (ReLU), as demonstrated below. However, the strictness of monotonicity may make a difference to the performance of the neural network. To illustrate this, the detailed experiments below consider two variants of LCN networks including different activation functions. These are denoted ReLU-LCN and Leaky-LCN. As the names suggest, the hidden layer activation functions of ReLU-LCN are all ReLU, while the hidden layer activations of Leaky-LCN are all leaky ReLU with α=0.3 in equation (3).

$$y = \{ \begin{matrix}x & if x > 0 \\ α x & if x \leq 0\end{matrix} \qquad ( 3 )$$

The Layer-Wise convex network (LCN) theorem is motivated by the aim to design neural networks rationally and to make the most out of the training set. A feed-forward neural network is essentially a computational graph where each layer can only “see” the layers directly connected to it, and has no way to tell whether its upstream layer is an input layer or a hidden layer. This “layer-unawareness” is similar to what is acknowledged in the development of batch normalisation [9] and is central to the LCN theorem. LCN approaches machine learning from function approximation and information theory perspectives, detailed below.

Suppose a training set of X∈ [symbol] ^{D×m} and training labels Y∈ [symbol] ⁿ, and that there exists a deterministic data generating process f: X [symbol] Y. The neural network aims to approximate the data generating process f. The universal approximation theorem [3], [7] states that a feed-forward neural network with linear output and at least one sufficiently wide hidden activation layer with a broad class of activation functions, including sigmoidal and piece-wise linear functions [14], can approximate any continuous function and its derivative [8] defined on a closed and bounded subset of [symbol] ⁿ to arbitrary precision.

The problem of neural network design is to determine how wide the hidden layer should be. According to universal approximation theorem, there exists a set of neural network parameters θ such that

$$\vert f - f ( θ ) \vert < ϵ \qquad ( 4 )$$

∀∈>0. As the neural network computes a chain of functions, if θ can be found, then ∀∈>0 and l∈[0, L] (i.e. the lth layer), and the neural network must satisfy the following equations:

$$\vert {g}^{[ l ]} ( {θ}^{[ l ]} {\tilde{A}}^{[ l - 1 ]} ) - {\tilde{A}}^{[ l ]} \vert < ϵ \qquad ( 5 )$$

$${A}^{[ 0 ]} = X \qquad ( 6 )$$

$$\vert {A}^{[ L ]} - Y \vert < ϵ \qquad ( 7 )$$

where Ã^{[l]}∈ [symbol] ⁽ⁿ^{^{[l]}}^{+1)×m}. Ã^{[l]} differs from A^{[l]} as it has one dummy row of is to include b into 0, i.e. Ã=[1; A].

To estimate θ, recall that an over-determined system of linear equations Ax=y has a unique set of solutions that minimises the Euclidean distance |Ax−y|₂. This property can be extended to nonlinear equations, as long as the nonlinear activation g^{[l]} is strictly monotonic and its reverse function is Lipschitz continuous. A real function h is said to be Lipschitz continuous if one can find a positive real constant K such that

$$\vert h ( {x}_{1} ) - h ( {x}_{2} ) \vert \leq K \vert {x}_{1} - {x}_{2} \vert \qquad ( 8 )$$

for any real x₁ and x₂ on the domain of h. Any function with bounded gradient on its domain is Lipschitz continuous. As the inverse function of a strictly monotonic function is defined and unique, the equivalent form of Eq. (8) can be written taking the inverse function of both sides:

$${g}^{- 1 [ l ]} ( {\tilde{A}}^{[ l ]} - ϵ ) < {θ}^{[ l ]} {\tilde{A}}^{[ l - 1 ]} < {g}^{- 1 [ l ]} ( {\tilde{A}}^{[ l ]} + ϵ ) \qquad ( 9 )$$

Using the Lipschitz continuity of g^{-1[l]}, a positive real constant K can be found such that

$${g}^{- 1 [ l ]} ( {\tilde{A}}^{[ l ]} ) - K ϵ \leq {g}^{- 1 [ l ]} ( {\tilde{A}}^{[ l ]} - ϵ ) < {θ}^{[ l ]} {\tilde{A}}^{[ l - 1 ]} < {g}^{- 1 [ l ]} ( {\tilde{A}}^{[ l ]} + ϵ ) \leq {g}^{- 1 [ l ]} ( {\tilde{A}}^{[ l ]} ) + K ϵ \qquad ( 10 )$$

∀∈, which implies

$$\vert {θ}^{[ l ]} {\tilde{A}}^{[ l - 1 ]} - {g}^{- 1} ( {\tilde{A}}^{[ l ]} ) \vert < K ϵ \qquad ( 11 )$$

$${lim}_{ϵ \to 0} {θ}^{[ l ]} {\tilde{A}}^{[ l - 1 ]} = {g}^{- 1} ( {\tilde{A}}^{[ l ]} ) \qquad ( 12 )$$

These equations conveniently transform the nonlinear equations (5) into a set of linear equations (12). The solution requires merely that (12) is over-determined, i.e. more equations are available than the number of variables. The input data set contains m training examples, each contributing to one equation. Therefore, the sufficient and necessary condition for equation (12) to have a unique solution that minimises the Euclidean distance |θÃ^{[L-1]}−g⁻¹(A^{[L]})|₂ is n_{θ}≤m. When n_{θ}=m the unique solution to make the Euclidean distance arbitrarily close to 0 can be found.

The Layer-Wise Convex Theorem can be stated as: For an L-layer feed-forward neural network, the sufficient conditions for there to exist a unique set of parameters W^{[l]} and b^{[l]} that minimises the Euclidean distance |A^{[l]}−g^{[l]}(W^{[l]}A^{[l-1]}+b^{[l]})|₂, ∀l∈[1, L] are:
n_{W}^{[l]}+n_{b}^{[l]}≤m, ∀l∈[0, L], where m is the number of training examples, and n_{W}^{[l]} and n_{b}^{[l]} are the number of weights and biases in layer l, respectively.
The network does not have skip connections;
All activation functions of the network are strictly monotonic, but different layers may have different monotonicity. For example, some layers can be strictly increasing, while other layers can be strictly decreasing.
All reverse functions of the activation functions are Lipschitz continuous.

A Layer-Wise Convex Network (LCN) is defined as any network fulfilling the Layer-Wise Convex Theorem.

Based on the above, a heuristic algorithm named AutoNet can be introduced, inspired by the reinforcement learning principle. The method is designed to automatically generate deep LCNs based on the characteristics of the input data, i.e. the training set. The method may provide a number of advantages over previous algorithms: (i) It monitors both training and validation losses to decide on the next step. (ii) It avoids dropout and does not add batch normalisation until the last step when growing the model, as both dropout and batch normalisation add much noise to the training process. (iii) By starting from a small model and grow the model to be just the right size for the problem, the algorithm avoids wasting computational resource in solving simple problems with huge models.

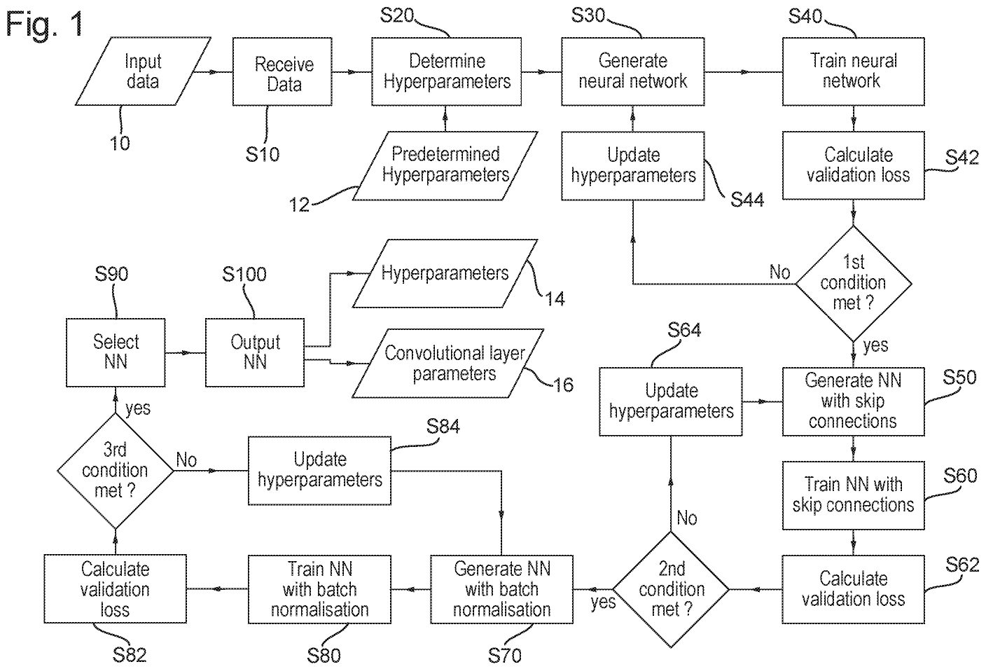

FIG. 1 shows an embodiment of a computer-implemented method for generating a neural network, of which the AutoNet algorithm is an example. The method comprises receiving S10 input data 10. In a preferred embodiment, the input data 10 comprises time series data. The time series data may comprise one or more channels of time-varying data, for example red, green, and blue colour channels of a two-dimensional (2D) video image. In some embodiments, the time series data is cyclic physiological data, for example electrocardiogram (ECG) data. ECG data is one-dimensional (1D), unlike the example of 2D video images, but may comprise multiple channels for the multiple leads of the ECG. In the CKB experiments discussed below, each training example in the input data 10 is 12-lead, 10 s, 500 Hz ECG time-series data. In that case, the input data 10 has 12 channels, and the dimension D of each training example is 5,000×12=60,000. While the application to cyclic physiological data such as ECG is shown in detail below, the method is also applicable to other input data 10.

The method comprises determining S20 values of a plurality of hyperparameters based on one or more properties of the input data 10 and generating S30, based on the values of the hyperparameters, a neural network comprising a plurality of layers. Some hyperparameters are determined and optimised by the method, as discussed below. However, there may be other hyperparameters on which the generated network is based that are determined from predetermined/default settings, and are held fixed in the method below. For example, default values may be used for pooling size (=2), learning rate, beta1 and beta2 of the Adam optimizer (discussed in the experiments section below) are not determined by AutoNet-LCN.

The hyperparameters determine the network architecture. In the embodiment of FIG. 1, the method generates a convolutional neural network (CNN) in which the plurality of layers comprises one or more pooling layers and one or more convolutional layers between each pooling layer. In this case, the plurality of hyperparameters comprises the number of pooling layers and the number of convolutional layers between each pooling layer. CNNs are networks with at least one layer of convolutional operation, and are an example of a weight sharing mechanism. The motivation for using a CNN is to reuse the “feature detectors” at multiple locations of the input data. For example, in an image processing application, the CNN should be able to detect eyes anywhere in the image. Another motivation behind CNNs is to share the weights within the same layer in order to reduce the number of parameters, effectively reducing overfitting and lower computational cost.

CNNs are not restricted to applications in image processing, and they can be applied to any input data that has distributed features. For example, the convolution operation can be performed on one-dimensional (1D) sequential data. Examples include ECG time-series data, which can be single-lead or multi-lead. Multiple ECG leads correspond to different channels, similar to the RGB channels of images. The difference from image applications is that nₕ=fₕ=1. Note that 1D CNN does not treat multi-channel sequential data as an image. In other words, using 1D CNN on multi-channel sequential data is not equivalent to stacking the channels together to form a 2D “image” and feeding the “image” into a 2D CNN. The former 1D approach requires the kernels of the first convolutional layer to have precisely ne channels, while the latter 2D approach allows for free choice of the kernel size along the nₕ dimension as long as fₕ≤n_{c}, while f_{c}=1. Here, in common with notation used in CNN for computer vision applications, nₕ is the height dimension of the input “image”; fₕ is the height dimension of the CNN kernel/filter; f_{w} is the width dimension of the CNN kernel/filter; f_{c} is the channel dimension of the CNN kernel/filter. The CNN kernel/filter is a cube with shape fₕ×f_{w}×f_{c}.

The values of the hyperparameters are determined based on one or more properties of the input data 10. The values of one or more of the hyperparameters may be predetermined, and the values of one or more of the other hyperparameters may be determined using the values of the predetermined hyperparameters. As discussed further below, the hyperparameters may comprise one or more of: i) the number of pooling layers; ii) the number of convolutional layers stacked between two pooling layers; and iii) the number of filters of each convolutional layer. In some embodiments, further neural network features which may be considered hyperparameters include whether skip connections are enabled, and whether batch normalisation is enabled.

Number of Pooling Layers, nₘₐₓₚₒₒₗ

A first hyperparameter that may be used to configure the neural network is the number of pooling layers nₘₐₓₚₒₒₗ. The number of pooling layers may be predetermined, preferably based on the properties of the input data. In the embodiments described below, the number of number of pooling layers is held fixed throughout the training process, but it is to be appreciated that in other embodiments the number of pooling layers may be varied at step S44 based on the outcome of step S42.

Pooling is often applied in CNNs, and involves calculating a value from every k input values, typically the max value or the mean value. Pooling in effect reduces the dimension of the resulting tensor. Pooling layers do not have parameters to learn. If the input tensor has n_{c} channels, the output of max-pooling also has n_{c} channels. The pooling is done on each channel independently.

Where the input data 10 is periodic time series data, the step S20 of determining values of the plurality of hyperparameters may comprise determining the number of pooling layers based on a number of samples in the time series data per period of the time series data. In the embodiment of FIG. 1, the hyperparameters comprise a predetermined estimate of the period of the time series data, also referred to as the timescale hyperparameter, and denoted τ. The hyperparameters further comprise a predetermined parameter quantifying a reduction in dimensionality by each pooling layer, also referred to as the pooling size, and denoted p. The number of pooling layers nₘₐₓₚₒₒₗ is determined according to Eq. (13)

nₘₐₓₚₒₒₗ=┌logₚ(fₛτ)┐ (13)

where fₛ is the sampling frequency of the time series data.

For example, the input may comprise 500 Hz ECG time-series data. Since ECG input data is highly periodic, with the duration of a heartbeat roughly once a second, the timescale hyperparameter is set to τ=1 s. The resulting neural network produces one prediction roughly every second. In general, it is desirable to use as small a pooling size as possible (i.e. p=2). This enables the generated networks to be as deep as possible. The number of pooling layers is thus ┌logₚ(500 Hz×1 s)┐=9.

In some embodiments, the input data 10 is non-periodic time series data. In such embodiments, in the step S20 of determining values of the plurality of hyperparameters, the number of pooling layers may be determined based on a number of samples in the time series data. This is essentially equivalent to setting fₛτ=D in Eq. (13), where D is the number of samples of the time series data, i.e., assuming that the entire input time-series represents one period. The hyperparameters still comprise the predetermined parameter quantifying a reduction in dimensionality by each pooling layer, also referred to as the pooling size, and denoted p. In this case, the network will output only one prediction for the entire signal, and the number of pooling layers nₘₐₓₚₒₒₗ is determined according to:

nₘₐₓₚₒₒₗ=┌logₚ(D)┐. (13a)

The pooling layers in some embodiments are max-pooling layers. Max-pooling is a pooling operation that calculates the maximum value in each patch of the feature map. Other embodiments use alternative pooling techniques, such as average pooling layers.

### Number of Filters in Each Convolutional Layer, n_{f}

A further hyperparameter used in the embodiments discussed below is the number of filters n_{f} in each convolutional layer. The number of filters may preferably be predetermined and held constant throughout the training process, but in some embodiments it may be varied at step S44 based on the outcome of step S42.

To consider the number of filters, it is useful to consider a concrete example of applying LCN theorem to design model architecture for the CKB dataset, which is a four-class classification task. Each training example in the dataset is a 12-lead, 10 s, 500 Hz ECG time-series, which means the input dimension D of each training example is 5000×12=60000. According to the LCN theorem, the number of parameters per layer should not exceed 6065. 6065 is the training size of the CKB dataset. Because D>m, if we use a feed-forward network, the first layer will have at least D parameters, thus we must use weight-sharing mechanisms, and CNN is a natural choice. This example is time-series data, and so 1-D CNN is a natural choice. In 1-D CNN, one of n_{w} and nₕ equals 1, and ne equals the number of input channels. In this work we use the convention nₕ=1, fₕ is also constrained to be 1. We use the letter k to denote f_{w}.

To simplify the design process, we use repeating structures and make sure all layers have the same output shape until the output layer. The repeating structure not only reduces the number of hyperparameters, but also is the least susceptible to vanishing and exploding gradient problems [4]. It is also easy to see that between the last convolutional layer and the output layer we should preferably not add fully connected layers. This is because in order not to exceed the upper bound, the dimension of densely-connected layers has to be very small. This would mean that it will become “bottlenecks” of the flow of information. Therefore it is preferable to only use convolutional, pooling (for dimension reduction because of 5,000×12×4+4>6,056), and softmax output layers. For CNN layers with kernel size k, stride s, padding p, and the number of filters n_{f}, the output shape of such convolutional layer is

$$( ⌊ \frac{input dimension - k + 2 p + 1}{s} ⌋ , {n}_{f} ) .$$

The number of parameters of this convolutional layer is n_{f}(kn_{f}+1) (assuming we are stacking several convolutional layers together). Since stride s>1 will result in dimension reduction, and empirically, it does not perform as well as max-pooling, in this example we keep s=1 (but some embodiments treat s as a hyperparameter). To keep output shape identical to the input shape, in this example we use “same” padding, then we calculate k and n_{f} by equations (17) and (18):

$$k = {n}_{f} = arg max {n}_{f} ( {n}_{f}^{2} + 1 ) \qquad ( 14 )$$

subject to:

$${n}_{f} ( {n}_{f}^{2} + 1 ) \leq m \qquad ( 15 )$$

We constrain k=n_{f} to avoid k being unreasonably large for long signals with few channels (but in other embodiments k is treated as a hyperparameter).
Number of Convolutional Layers, nᵣₑₚₑₐₜ

A further hyperparameter is the number of convolutional layers between max-pooling layers, nᵣₑₚₑₐₜ. There are no guidelines to calculate the optimal depth of the convolutional layers, and so no optimal or near-optimal value that can be initially assigned to nᵣₑₚₑₐₜ. In the examples below, therefore, nᵣₑₚₑₐₜ is initially set to 1 (i.e. one convolutional layer between each pair of pooling layers). nᵣₑₚₑₐₜ is then varied incrementally at step S44 to refine the neural network. The general principal is that adding layers should not harm performance, although the training may become more difficult.

### Skip and Batch Normalisation

As will be described further below, further factors which may be considered as hyperparameters and which are used in some embodiments include whether skip and batch normalisation are used. These factors act as switches, turning on skip connections or batch normalisation. When used, these factors are initial set to off.

### Generating the Baseline Neural Network

Having determined the initial hyperparameters at step S20, the method of FIG. 1 then moves to step S30. At step S30, a baseline neural network is generated using the initial hyperparameters.

An example algorithm for generating a baseline LCN neural network is shown in Algorithm 1 below. This example uses the five hyperparameters discussed above, nᵣₑₚₑₐₜ∈ [symbol] , nₘₐₓₚₒₒₗ∈ [symbol] , n_{f}∈ [symbol] , skip∈B (Boolean domain), and bn∈B. The number of filters n_{f} is the is calculated according to equations (14) and (15). The number of max-pooling layers nₘₐₓₚₒₒₗ is determined according to equation (13) or (13a). The output layer is a time-distributed softmax layer for classification and classifies the entire signal by majority voting. skip and bn are the “switches” representing whether the network adds skip connections and batch normalisation, respectively.

The number of convolutional layers preceding each pooling layer, nᵣₑₚₑₐₜ is initially set to 1. As will be appreciated, an activation layer may be placed between each convolutional layer and pooling layer. The activation layer may comprise a rectified linear unit (ReLU) or a leaky rectified linear unit (leaky ReLU). For example, the hidden layer activations of Leaky-LCN may be leaky ReLU with α=0.3 in equation (3).

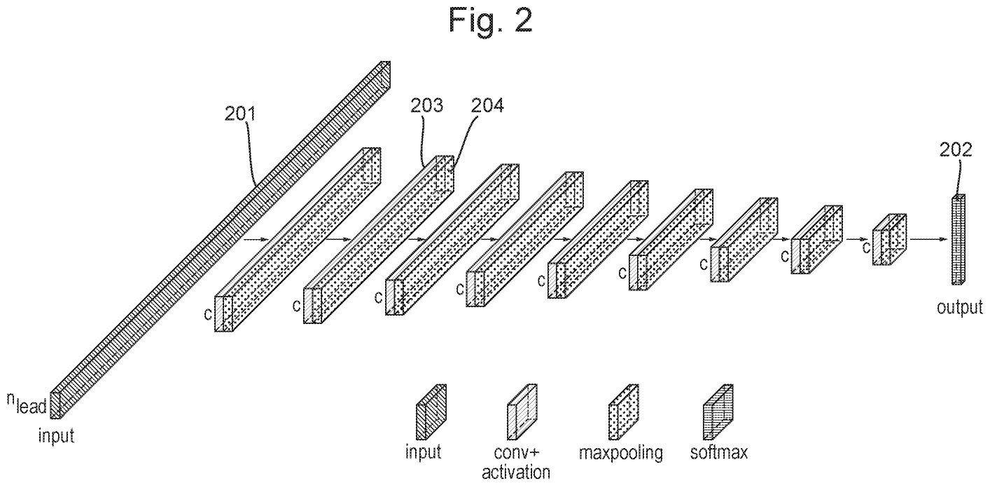

FIG. 2 illustrates the baseline architecture generated by Algorithm 1 for the case nₘₐₓₚₒₒₗ=9 (as for the CKB dataset discussed above). In FIG. 2, the neural network comprises an input layer 201, and an output layer 202. The output layer may include a classifier layer. Between the input layer 201 and output layer 202 are a number of convolutional layers 203 and pooling layers 204. For clarity only one of each of the convolutional layers 203 and pooling layers 204 are labelled, but the repeating pattern of one convolutional layer 203 preceding each pooling layer 204 is clearly visible. In FIG. 2, the activation layer is incorporated into convolutional layer 203.

### Training the Neural Network

Having generated the baseline neural network, the method of FIG. 1 proceeds to step S40, at which the baseline neural network is trained using the input data 10.

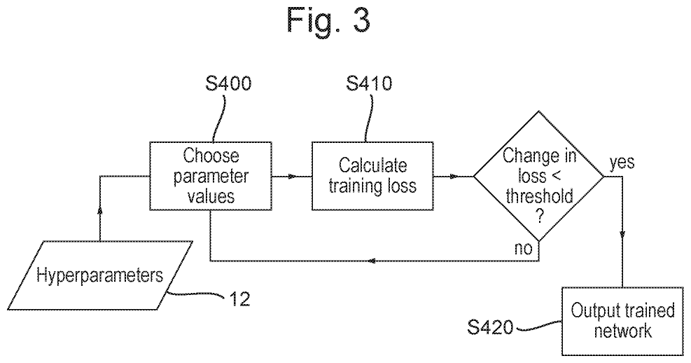

FIG. 3 illustrates an example method for training the neural network, which may be used as step S40 in FIG. 1. In this example, the input data 10 is labelled input data, and the neural network is trained using supervised learning. This method may be used in embodiments in which a CNN is generated using hyperparameters 12 including the number of pooling layers nₘₐₓₚₒₒₗ and the number of convolutional layers nᵣₑₚₑₐₜ between each pooling layer. Each convolutional layer has an associated plurality of

ALGORITHM 1

| | Input: m, n_{channel}, n_{class}, nᵣₑₚₑₐₜ, skip, bn, | | | |
|---|---|---|---|---|
| | | nₘₐₓₚₒₒₗ. | | |
| | Output: model. | | | |
| 1 | n_{f} = argmaxₙ_{_{f}}n_{f}(n_{f}² + 1) subject to | | | |
| | n_{f}(n_{f}² + 1) ≤ m. | | | |
| 2 | add the input layer. | | | |
| 3 | if bn then | | | |
| 4 | \| | add a batch normalisation layer. | | |
| 5 | end | | | |
| 6 | add a convolutional layer, kernel size = n_{f}, | | | |
| | n_{filters} = n_{f}. | | | |
| 7 | if bn then | | | |
| 8 | \| | add a batch normalisation layer. | | |
| 9 | end | | | |
| 10 | add a maxpooling layer, pooling size= 2. | | | |
| 11 | for _ in range nₘₐₓₚₒₒₗ − 1 do | | | |
| 12 | \| | for _ in range nᵣₑₚₑₐₜ do | | |
| 13 | \| | \| | add a convolutional layer, kernel size = n_{f}, | |
| | \| | \| | n_{filter} = n_{f}. | |
| 14 | \| | \| | if skip then | |
| 15 | \| | \| | \| | connect the before-activation output of |
| | \| | \| | \| | every nₘₐₓₚₒₒₗ − 1 convolutional layers |
| | \| | \| | \| | by addition. |
| 16 | \| | \| | end | |
| 17 | \| | \| | add an activation (ReLU or leaky ReLU) | |
| | \| | \| | layer. | |
| 18 | \| | \| | if bn then | |
| 19 | \| | \| | \| | add a batchnorm layer. |

| | | | |
|---|---|---|---|
| 20 | \| | \| | end |

| | | |
|---|---|---|
| 21 | \| | end |
| 22 | \| | add a maxpooling layer. |

| | |
|---|---|
| 23 | end |
| 24 | add a time distributed softmax layer. |

parameters.

In some embodiments, the input data is physiological data. The neural network may be constructed to include a classification layer configured to classify the input data into one of a plurality of clinical categories.

The method of FIG. 3 starts at step S400, at which values of the parameters of the convolutional layers are chosen based on the values of the hyperparameters 12 and selected initial (or for repeat loops, previous) values of the parameters of the convolutional layers.

At step S410, a training value of a loss function is calculated using an output of the neural network.

Steps S400 is then repeated to vary the parameters. A new training value of the loss function is calculated at step S410. The change in the training value of the loss function is compared to the previous cycle is then compared to a predetermined threshold.

The steps S400 and S410 are further repeated until the change in the training value of the loss function over two or more consecutive steps of calculating the training value of the loss function is below a predetermined threshold. When the change in training value of the loss function is below the predetermined threshold, the trained network is output at step S420. Outputting the trained network may comprise outputting the parameters of the chosen in the final repetion of step S400. The trained network is then used in the next steps of the method of FIG. 1.

In some embodiments, the training value of the loss function comprises a training loss calculated by evaluating the loss function on the output of the neural network applied to the input data. In general, the choices of the loss functions and the output activation functions are closely linked to the machine learning problem. For binary classification the preferred choice is the binary cross-entropy loss with a sigmoid output in Eq. (16); for K-class (K>2) classification the preferred choice is the multi-class cross-entropy loss with a softmax output in Eq. (17); and for regression problems, the preferred choice is the mean squared error and linear output (identity mapping) in Eq. (18).

$$E = - \frac{1}{m} {\sum }_{i = 1}^{m} [ {y}_{i} log {\hat{y}}_{i} + ( 1 - {y}_{i} ) log ( 1 - {\hat{y}}_{i} ) ] \qquad ( 16 )$$

$$E = - \frac{1}{m} {\sum }_{i = 1}^{m} {\sum }_{k}^{K} {y}_{ik} log {\hat{y}}_{ik} \qquad ( 17 )$$

$$E = \frac{1}{m} {\sum }_{i = 1}^{m} {( {y}_{i} - {\hat{y}}_{i} )}^{2} \qquad ( 18 )$$

### Updating the Hyperparameters

Once the parameters for the initial neural network have been optimised, the method of FIG. 1 proceeds to determine if a first predetermined condition is met. If the first condition is not met, the hyperparameters of the neural network are updated.

In the illustrated example, at step S42 a validation value of a loss function is calculated for model trained in step S40. The validation value of the loss function may comprise a validation loss calculated by evaluating the loss function on the output of the neural network applied to a validation data set. The first predetermined condition is met if the validation value is not lower than the validation value of the loss function of the neural network following the training of the previous neural network. In this embodiment, the first predetermined condition cannot be met after just the training of the initial neural network. In such cases, the method always proceeds to step S44 after completing step S42 for the initial neural network. 20. The loss function used for validation may be same the same as that for the training in step S410. For example, one of the equations (16)-(18) may be used as the loss function. Alternatively a different loss function may be used for hyperparameter validation.

At step S44, the value of one or more of the hyperparameters is updated. Preferably, only one hyperparameter is changed. The loop of steps S30-S44 can be run to optimise that one hyperparameter, before then updating and optimising a different hyperparameter.

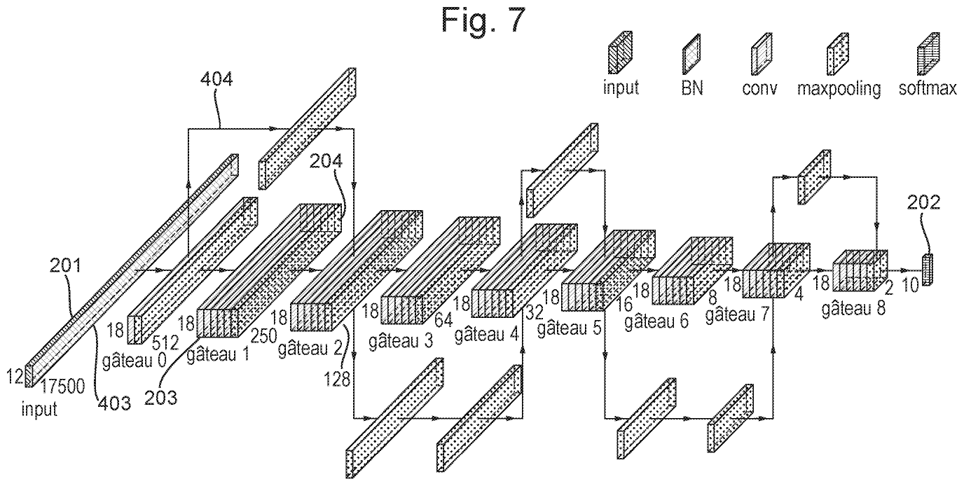

In particular embodiments, the number of convolutional layers between pairs of pooling layers, nᵣₑₚₑₐₜ is the varied hyperparameter. Step S44 may comprise incrementing nᵣₑₚₑₐₜ by one compared to its previous value. Alternatively, nᵣₑₚₑₐₜ may be incremented by a higher integer. As shown for example in FIG. 7, there may always be one convolutional layer between the input layer and the first pooling layer. The varying of the hyperparameter nᵣₑₚₑₐₜ (the number of convolutional layers between pooling layers) does not affect the number of convolutional layers between the input layer and the first pooling layer.

Once the hyperparameter/s have been updated, an updated neural network is generated at step S30 based on the updated hyperparameters. For example, Algorithm 1 may be used to generate the updated neural network. The updated neural network is then trained in step S40 to optimise its parameters. An updated validation value of the loss function is determined at step S42 for the trained updated network. The updated validation value is compared to the previous validation value to determine if the first condition is met. If the first condition is not met, the method repeats steps S44, S30, S40, and S42 for a further updated set of hyperparameters (e.g. incrementing nᵣₑₚₑₐₜ by one again).

This cycle of updating hyperparameters and generating and training a network based on those hyperparameters continues until the first predetermined condition is met. The first predetermined condition may be met when there is no reduction in the validation loss for a predetermined number of cycles/epochs (i.e. loops of steps S30-S44). The predetermined number may be in the range 2-15, or 5-10. Preferably the predetermined number is 8.

In some embodiments, the first predetermined condition is only met when there is no reduction in validation loss or training loss for the predetermined number of epochs. In other words, even if there is no reduction in the validation value of the loss function calculated in step S42 compared to the previous epoch, the first condition still won't be met if the training value of the (training) loss function is reduced compared to the previous epoch.

Once the first predetermined condition has been met, some embodiments output the optimised neural network for use to train real world data. This may comprise storing, transmitting or otherwise outputting the optimised values of the hyperparameters. The optimised hyperparameters may be the hyperparameters used for the network when the first condition was met. The optimised hyperparameters may be the hyperparameters used for the neural network with the lowest validation value. The trained parameters of the convolutional layers of the neural network with the optimised hyperparameters may also be output. Outputting may comprise performing steps S90 and S100 discussed in more detail below.

Alternatively, some embodiments continue to refine the neural network by introducing skip connections and/or batch normalisation, as illustrated in FIG. 1.

### Enabling Skip Connections

Once the hyperparameters have been optimised as described above, the method illustrated in FIG. 1 then optionally proceeds to step S50. At step S50, skip connections are enabled.

Skip connections are also called residual connections. Skip connections are a way to address the vanishing gradient problem in training deep networks. They work by copying the activations of a far-away layer to the current layer. The addition is performed originally before activation and after the affine transformation (equation (19)), where the residual connection connects layer l and layer l−δ), although there are many variations. One example is “ResNet”, developed by He, K. et al. [6], which is incorporated herein by reference. ResNet has 152 layers and 60 million parameters.

$${A}^{[ l ]} = g ( {W}^{[ l ]} {A}^{[ l ]} + {b}^{[ l ]} + {A}^{[ l - δ ]} ) \qquad ( 19 )$$

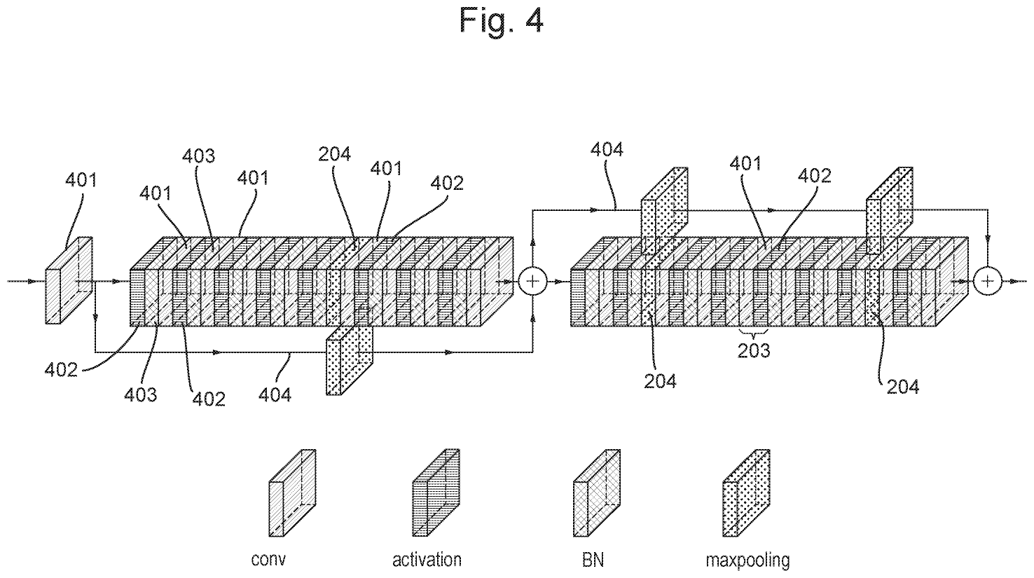

At step S50, the method generates a neural network based on the optimised hyperparameters from steps S44, S30, S40, and S42, but with skip connections between non-consecutive layers of the neural network. In some embodiments, the skip connections connect every (nₘₐₓₚₒₒₗ−1)^{th} layer by adding the convolutional output of the (l−(nₘₐₓₚₒₒₗ−1))^{th} layer to the convolution output l^{th} convolutional layer. The element-wise addition is applied to the output of the convolution stage of a convolutional layer, before the activation is applied. So for example, where nₘₐₓₚₒₒₗ=9, the output tensor (pre-activation) of the first convolutional layer is added to the convolution output (pre-activation) of the ninth convolutional layer. The output tensor (pre-activation) of the ninth convolutional layer is likewise added to the convolution output of the seventeenth convolutional layer, and so on. An example of a skip connection 404 is shown in FIG. 4, discussed below. One or more pooling layers may be applied to the output of the (l−(nₘₐₓₚₒₒₗ−1))^{th} layer as part of the skip connection to ensure the data size matches that of the later layer. The number of pooling layers applied to a skip connection may match the number of pooling layers in the non-skipped path between the (l−(nₘₐₓₚₒₒₗ−1))^{th} and lth layers.

Having generated the neural network with skip connections, the method proceeds to steps S60. At step S60 the generated neural network is trained to optimise its parameters. Step S60 is substantially the same as step S40 discussed above. Step S60 may use the method of FIG. 3.

The method then determines if a second predetermined condition is met, and either updates the hyperparameters or outputs the hyperparameters accordingly. In the illustrated example, the second predetermined condition is met when a validation value of a loss function of the neural network comprising one or more skip connections following the step of training the neural network comprising one or more skip connections is not lower than the validation value of the loss function of the neural network comprising one or more skip connections following the training of the previous neural network comprising one or more skip connections. To this end, the illustrated method proceeds to step S62. At step S62 a validation loss function is calculated for the trained neural network. Step S62 is substantially similar to step S42 discussed above. The method then determines whether the second predetermined condition is met.

As with the determination as to whether the first predetermined condition is met, the second predetermined condition may only be met when there is no reduction in the validation loss for a predetermined number of cycles/epochs (i.e. loops of steps S30-S44). The predetermined number may be in the range 2-15, or 5-10. Preferably the predetermined number is 8. In some embodiments, the second predetermined condition is only met when there is no reduction in validation loss or training loss for the predetermined number of epochs.

If the second predetermined condition is not met, the method proceeds to step S64. At step S64 one or more of the hyperparameters is updated, similar to the process in step S44. In particular examples, updating the hyperparameters comprises updating the number of convolutional layers between pairs of pooling layers, nᵣₑₚₑₐₜ. In some embodiments, step S64 comprises incrementing nᵣₑₚₑₐₜ compared to its previous value by an increment amount. The increment amount may be 1, or any other predetermined increment.

The method then returns to step S50, at which an updated neural network is generated based on the updated hyperparameters, with the skip connections discussed above enabled. Steps S60 and S62 are performed to train the updated network, calculate a validation value of the loss function, and determine if the second predetermined condition is met. The method continues to loop through steps S64, S50, S60, S62 until the second predetermined condition is met.

Once the second predetermined condition is met, some embodiments may output the results for use in training real world data, as discussed above in relation to meeting the first predetermined condition. In the illustrated embodiment, however, the method performs a further optimisation stage by enabling batch normalisation.

### Enabling Batch Normalisation

In the illustrated embodiment, the method then proceeds to step S70, at which batch normalisation is enabled. Batch normalisation is used to reduce internal covariate shift, and is discussed in Ioffe, S., et al. [9], which is incorporated herein by reference. Batch normalisation has analogous effect as normalising the input features to machine learning models, the key difference being that the batch normalisation normalises the hidden layer outputs rather than the input data. This results in improved Hessian conditioning which facilitates optimization, similar to how normalising the input features improves Hessian conditioning of machine learning models with quadratic loss (e.g. linear regression with mean squared error loss). At step S70, a neural network is generated based on the optimised hyperparameters output by the preceding stage of the method (i.e. loop S30, S40, S42, S44 or loop S50, S60, S62, S64). The neural network generated in step S60 is generated with one or more batch normalisation layers. In some embodiments, a batch normalisation layer is added after each activation layer. A batch normalisation layer may also be added after an input layer.

The method then proceeds to step S80, where the newly generated neural network is trained. Step S80 is similar to steps S40 and S60 discussed above. Step S80 may use the method of FIG. 3. It is then determined if a third predetermined condition is met. In the illustrated embodiment, the method proceeds to calculate a validation value of a loss function at step S82 (similar to steps S42 and S62). The third predetermined condition is met when the validation value of a loss function of the neural network comprising one or more batch normalisation layers following the step of training the neural network comprising one or more batch normalisation layers is not lower than the validation value of the loss function of the neural network comprising one or more batch normalisation layers following the previous step of training the neural network comprising one or more batch normalisation layers. The loss function may be the same as or different to the loss functions used for validation in steps S42 and S62.

As with the determination as to whether the first and second predetermined conditions are met, the third predetermined condition may only be met when there is no reduction in the validation loss for a predetermined number of cycles/epochs (i.e. loops of steps S30-S44). The predetermined number may be in the range 2-15, or 5-10. Preferably the predetermined number is 8. In some embodiments, the second predetermined condition is only met when there is no reduction in validation loss or training loss for the predetermined number of epochs.

If the third predetermined condition is not met, the method proceeds to step S84. At step S84, one or more of the hyperparameters is updated, similar to the process in steps S44 and S64. In particular examples, updating the hyperparameters may comprise updating the number of convolutional layers between pairs of pooling layers, nᵣₑₚₑₐₜ. In some embodiments, step S64 comprises incrementing nᵣₑₚₑₐₜ compared to its previous value by an increment amount. The increment amount may be 1, or any other predetermined increment.

The method then proceeds to step S70, at which an updated neural network is generated based on the updated hyperparameters, and with the one or more batch normalisation layers discussed above. The updated network is trained as step S80, and a validation loss calculated at step S82 for determination as to whether the third predetermined condition is met. This process is repeated until the third predetermined condition is met.

In the illustrated embodiment, the hyperparameter optimisation stages are now complete. However, other embodiments may comprise further optimisation stages for particular hyperparameters or hyperparameter-like factors. The skilled person will appreciate that the number of stages of optimisation may be selected based on the type of network being optimised (e.g. LCN).

### Selecting and Outputting the Optimised Neural Network

Once the third predetermined condition is met, the illustrated method proceeds to step S90. At step S90, one of the trained neural networks is selected to be output. The selected trained neural network may be a neural network trained at any of steps S40, S60, or S80. In other words, there is no requirement to select a neural network with skip connections and/or batch normalisation enabled.

In some embodiments selecting one of the trained neural networks comprises selecting the trained neural network having a lowest validation value of a loss function. The model which yields minimum validation loss is taken to be the “best” model. In some embodiments, the validation value of the loss function comprises a validation loss calculated by evaluating the loss function on the output of the trained neural network applied to a validation data set, which may be different to the input data set 10.

Once the “best” hyperparameter model has been identified, the parameters of the convolutional layers of that “best” model may be further refined. In particular, some embodiments train the selected “best” neural network a plurality of times to obtain a corresponding plurality of trained instances of the “best” neural network. Training may use the method of FIG. 3. An average ensemble of the trained instances is then provided as the selected and output neural network. For example, the identified “best” network architecture may be trained K times. At the test time, the average probability predictions provided by the K models is calculated. The test case is then classified to the class with the highest mean probability, i.e.

$$\hat{i} = {arg max}_{i} \frac{1}{K} {\sum }_{j = 1}^{K} {p}_{ij} \qquad ( 20 )$$

where pᵢⱼ is i-th class's probability predicted by the j-th model. This step can be omitted if one is not reporting the final results and wishes to prototype quickly. Intuitively, the predicted probabilities of each of the K models are averaged, and the test case is classified as the class which has the highest average probability.

Having selected (and optionally further trained) the “best” network, the method proceeds to step 100. At step S100 the selected neural network is output. Outputting may comprise outputting the hyperparameters 14 of the selected network. Outputting may additionally comprise outputting the values of the parameters 16 of the convolutional layers of the selected network. The output hyperparameters 14 and/or parameters 16 may be stored or transmitted or otherwise output for use with sample data.

Algorithm 2, shown below, illustrates an algorithm that may be used to perform the method steps discussed above. Algorithm 2 calls Algorithm 1 to build each LCN, then trains the model until early stopping criteria is met. It tracks the minimum training loss and the minimum validation loss during training and compare them against the policy.

FIG. 4 illustrates the architecture of part of a neural network that may be generated by Algorithm 2. FIG. 4 shows the positions of convolutional 401, activation 402, batch normalisation 403, max-pooling layers 204, and the skip connections 404. In FIG. 4 the convolutional layers 401 and activation layers 402 are shown separately so that the skip connections can be illustrated. A convolutional layer 401 and its activation layer 402 together correspond to the convolutional (+activation) layers 203 shown in FIG. 2. For clarity, only some layers are labelled in FIG. 4.

ALGORITHM 2

| | Input: m, n_{channel}, n_{class}, nᵣₑₚₑₐₜ, skip, bn, | |
|---|---|---|
| | | nₘₐₓₚₒₒₗ, X, Y, model_averaging, |
| | | fold = 10. |
| | Output: best model. | |
| 1 | batch size = 32, patience = 8, bn = False, skip = | |
| | False. | |
| 2 | build a LCN model using Algorithm 1 and train it. | |
| 3 | while min_train_loss or min_validation_loss declines | |
| | do | |
| 4 | \| | nᵣₑₚₑₐₜ = nᵣₑₚₑₐₜ + 1. |
| 5 | \| | build a new LCN using Algorithm 1 and train it. |
| 6 | \| update min_train_loss and min_validation_loss. | |
| 7 | end | |
| 8 | skip =True. | |
| 9 | while min_train_loss or min_validation_loss declines | |
| | do | |
| 10 | \| | nᵣₑₚₑₐₜ = nᵣₑₚₑₐₜ + 1. |
| 11 | \| | build a new LCN using Algorithm 1 and train it. |
| 12 | \| | update min train loss and min_validation_loss. |
| 13 | end | |
| 14 | bn =True. | |
| 15 | while min_train_loss or min_validation_loss declines | |
| | do | |
| 16 | \| | nᵣₑₚₑₐₜ = nᵣₑₚₑₐₜ + 1. |
| 17 | \| | build a new LCN using Algorithm 1 and train it. |
| 18 | \| | update min_train_loss and min_validation_loss. |
| 19 | end | |
| 20 | best_model = the model with min_validation_loss. | |
| 21 | if model_average then | |
| 22 | \| | train the best network fold times. |
| 23 | \| | best_model = the average ensemble of the fold |
| | \| | models. |

| | |
|---|---|
| 24 | end |

The illustrated network has convolution-activation-BN repeating structure, with nₘₐₓₚₒₒₗ=9, nᵣₑₚₑₐₜ=5. A max-pooling layer is added after every nᵣₑₚₑₐₜ (5 in this example) batch normalisation layers. The element-wise addition for the skip connection is applied to the output tensor of every nₘₐₓₚₒₒₗ−1 (8 in this example) convolutional layers. For example, the output tensor of the first convolutional layer is elementwisely added to the output tensor of the 9th convolutional layer, and the resulting tensor is the input to the following activation layer and is also used in the element-wise addition with the output tensor of the 17^{th} convolutional layer. A pooling layer 204 is applied to the skip connection 404 to reduce the dimensions of the inputs, matching the reduction applied to the non-skipped path.

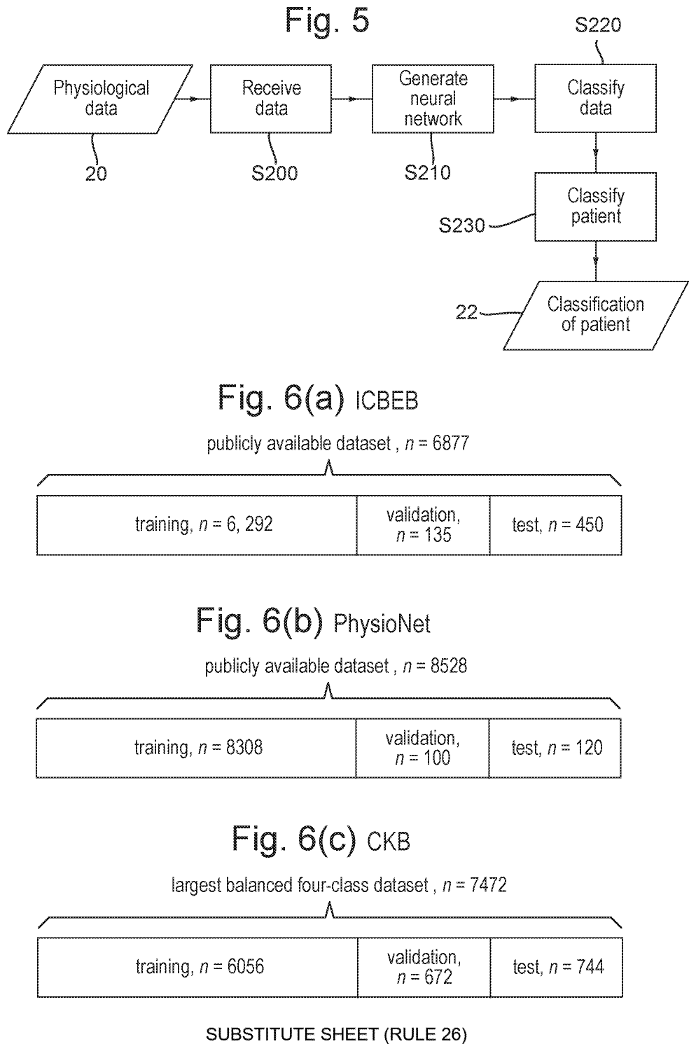

FIG. 5 illustrates an example method for using a network generated by the method of FIG. 1 to classify physiological data. The method of FIG. 5 starts at step S200, where physiological data 20 is received. The physiological data may be data measured from one or more patients. Receiving the physiological data may comprise retrieving stored physiological data. The method may also comprise measuring the physiological data. The method may be performed online, as the data is received, e.g. from electrodes attached to a patient.

The method the proceeds to step S210, at which a neural network is generated. Step S210 may comprise performing the method of FIG. 1. Alternatively, step S210 may comprise retrieving the hyperparameters 14 and convolutional parameters 16 output in step S100 of FIG. 1.

The method then proceeds to step S220, at which the physiological data 20 is classified by the generated neural network. Optionally, the method may then proceed to step S230, at which the patient is classified into one of a plurality of clinical categories based on the classification of the physiological data from the classification layer of the neural network. The classification of the patient 22 is then output for use by a clinician. For example, the clinical categories may include one or more of arrhythmia, ischemia, hypertrophy, normal individual.

As will be appreciated, the methods of FIGS. 1, 3, and 5 may be implemented as computer-executable instructions which, when the program is executed by a computer, cause the computer to carry out the method of any of the preceding claims. The instructions may be stored in a transient or non-transient computer-readable medium. For example, the instructions may be stored in a memory associated with the computer executing the instructions. The methods may be implemented by an apparatus for generating a machine-learning network, the apparatus comprising a receiving unit and a processing unit. The receiving unit is configured to receive input data comprising time series data. The processing unit is configured to: determine values of a plurality of hyperparameters based on one or more properties of the input data; generate, based on the values of the hyperparameters, a convolutional neural network comprising a plurality of layers; train the neural network using the input data and, at least if a first predetermined condition is not met, updating the values of one or more of the hyperparameters; repeat the steps of generating a neural network, and training the neural network until the first predetermined condition is met; select one of the trained neural networks; and output the selected neural network.

### Experiments

The method described above (i.e. the AutoNet algorithm, as shown in Algorithm 2) was used to generate networks for classifying electrocardiogram (ECG) data. The AutoNet-generated LCNs were demonstrated to perform at least as well as the state-of-the-art end-to-end deep learning model, with no more than 2% of the parameters, and the architecture search time is no more than 2 hours.

Performance of auto-generated LCNs compared to the state-of-the-art deep learning model for ECG classification was tested on three datasets: (i) International Conference on Biomedical Engineering and Biotechnology (ICBEB) (http://2018.icbeb.org/Challenge.html) Physiological Signal Challenge 2018, (ii) the PhysioNet Atrial Fibrillation Detection Challenge 2017 [2], and (iii) the China Kadoorie Biobank (CKB) (https://www.ckbiobank.org/site/). LCNs generated by AutoNet were benchmarked with the ResNet-based Hannun-Rajpurkar model [5], [15] which has been demonstrated to exceed average cardiologist performance in classifying 12 rhythm classes on 91,232 recordings from 53,549 patients and is well regarded as the state-of-the-art.

### Datasets

1) ICBEB Dataset: The publicly available training set of International Conference on Biomedical Engineering and Biotechnology (ICBEB) 2018 challenge includes 12-lead 500 Hz 5-143 s ECG time-series waveform from 6,877 participants (3,178 female and 3,699 male) obtained from 11 hospitals (http://2018.icbeb.org/Challenge.html). The dataset has nine classes. The primary evaluation criterion of the Challenge is the 9-class average F₁, calculated as equation (21) The secondary evaluation criteria are F₁ scores of sub-abnormal classes: F_{AF}, F_{Block}, F_{PC}, F_{ST} calculated as equations (22), (23), (24) and (25).

$${F}_{1} = \frac{1}{9} {\sum }_{i = 1}^{9} \frac{2 {N}_{ii}}{{\sum }_{j = 1}^{9} ( {N}_{ij} + {N}_{ji} )} \qquad ( 21 )$$

$${F}_{AF} = \frac{2 {N}_{22}}{{\sum }_{j = 1}^{9} ( {N}_{2 j} + {N}_{j 2} )} \qquad ( 22 )$$

$${F}_{Block} = \frac{2 {\sum }_{i = 3}^{5} {N}_{ii}}{{\sum }_{i = 3}^{5} {\sum }_{j = 1}^{9} ( {N}_{ij} + {N}_{ji} )} \qquad ( 23 )$$

$${F}_{PC} = \frac{2 {\sum }_{i = 6}^{7} {N}_{ii}}{{\sum }_{i = 6}^{7} {\sum }_{j = 1}^{9} ( {N}_{ij} + {N}_{ji} )} \qquad ( 24 )$$

$${F}_{ST} = \frac{2 {\sum }_{i = 8}^{9} {N}_{ii}}{{\sum }_{i = 8}^{9} {\sum }_{j = 1}^{9} ( {N}_{ij} + {N}_{ji} )} \qquad ( 25 )$$

2) PhysioNet Dataset: The publicly available training set of the PhysioNet 2017 Atrial Fibrillation Detection Challenge [2] (incorporated herein by reference) has 8,528 recordings, 9-60 s in duration, 300 Hz, single-lead ECG acquired using AliveCor. The dataset has four classes: 5,050 normal recordings, 738 atrial fibrillation recordings 2,456 “other rhythms” recordings, and 284 noisy recordings. The numbers are counted from the downloaded dataset, which is very different from what is stated on the website.

3) CKB Dataset: The China Kadoorie Biobank (CKB) [1] is publicly available at http://www.ckbiobank.org/site/Data+Access. For 24,959 participants, a standard 12-leadECG (10-s duration, sampled at 500 Hz) was recorded. After removing 113 participants with incomplete records, the remaining 24,906 participants were grouped in the five classes.

The train-validation-test split of each dataset used in the experiments is shown in FIG. 6.

### Experimental Configuration

1) Model Training: All LCN models were trained using Adam with default hyperparameters (β₁=0.9, β₂=0.999) and the default learning rate (0.001). Adam is described in Kingma, D. P., et al., [11], which is incorporated herein by reference. The Hannun-Rajpurkar model, as a bench-marking approach, was trained using the authors' original implementation (https://zithubcom/awni/eca) to ensure identical implementation. In brief, the Hannun-Rajpurkar model used Adam [11] with learning rate scheduler that decreases learning rate after no improvement on the validation loss for two epochs. All hyperparameters were kept the same as in his codes and as described in [5]. All models were trained using early stopping with patience 8 epochs, for a maximum of 100 epochs, which is the same as in Hannun's codes and in [5]. All experiments were performed on Ubuntu 18.04, CPU with 32 GB RAM, single Nvidia GeForce GTX 1080 GPU, with Python version 2.7.15, and Tensorflow version 1.8.0.

2) Power Analysis: To detect statistical significance a power analysis was conducted for the two-tail paired, t-test at effect size 0.8, α=0.05, power=0.8, and the required sample size was found to be 14.30. Therefore we conducted five repeats for each of the ICBEB, PhysioNet, and CKB, producing 15 experiments in total. In each repeat, all models were trained and tested on the same training, validation, and test sets. Note that the paired t-test only assumes the differences of the means, rather than the samples themselves, follow a Gaussian distribution, and does not assume equal variance of the samples [10]. Therefore the 15 experiments created by five repeats on three datasets are appropriate for the two-tail paired t-test, if the differences of the means pass normality tests.

### C. ICBEBDataset

1) Train-Validation-Test Split: We did not have access to the hidden test set, therefore we randomly took 50 samples from each class from the publicly available training set (n=6,877) to build a balanced test set (n=450) of the same size and class distribution as the ICBEB Challenge, another 15 samples per class to form a balanced validation set. FIG. 6(a) summarises the split details. We repeated the split and experiment five times. In each repeat, all models share the same training, validation, and test sets.

2) Sample Weighting: The samples in the training set (excluding the validation samples) were weighted by the inverse of their class ratio in the training set. For example, if class i has nᵢ samples in the training set, then each sample of class i receives

$$\frac{{\sum }_{i} {n}_{i}}{{n}_{i}}$$

weight during training.

3) Signal Padding: Since the pooling size is fixed in both LCC and Hannun-Rajpurkar models during training, these models require the input signal to have the same length. Ideally, the target length should be the maximum signal length in the training set, i.e. 61 s. However, due to memory constraints, we could only feed in 37 s signals. Thus the target length for ICBEB is 37 s. If the original signal was shorter than the target length, 0 s are padded to the end of the signal; if the signal is longer than the target length, the end of the signal was truncated. At test time, no padding is needed as the model generates a label every 512 time steps (1.024 s).

4) Model Generation: In each repeat, AutoNet identifies the “best” ReLU-LCN model and the “best” Leaky-LCN model separately. The hyperparameter f is calculated according to equations (14) and (15) with m=6,292, thus n_{f}=20. nₘₐₓₚₒₒₗ is calculated according to equation (13) with fₛ=500 Hz, τ=1 s, p=2, to be 9. It took AutoNet 1 h 25 min (5,095 s) on average to identify the best ReLU-LCN model and 1 h 55 min (6,936 s) to identify the best Leaky-LCN model. For ReLU-LCN, three out of five repeats converged at nᵣₑₚₑₐₜ=5 with both skip connections and batch normalisation (FIG. 7), one experiment converged at nᵣₑₚₑₐₜ=6, with both skip connections and batch normalisation, one experiment converged at nᵣₑₚₑₐₜ=4, with both skip connections and batch normalisation (Table I); for Leaky-LCN, four out of five repeats converged at nᵣₑₚₑₐₜ=5, with both skip connections and batch normalisation, while the other repeat converged at nᵣₑₚₑₐₜ=7, with both skip connections and batch normalisation.

FIG. 7 shows a visualisation of the auto-generated ReLU-LCN for ICBEB: nᵣₑₚₑₐₜ=5, nₘₐₓₚₒₒₗ=9, meaning there are a total of 9 max-pooling layers 204, and there are five convolutional layers 203 stacked between every two max-pooling layers 204. A batch normalisation layer 403 is added after the input layer 201 and after each convolutional (+activation) layer 203. Only one batch normalisation layer 203 is illustrated to declutter the figure. The skip after-convolution tensor is added to every 8 subsequent after-convolutional tensors, which are labelled in the figure. The output layer is a time-distributed 10-unit softmax layer, one unit for each of the nine classes and one unit to indicate noise/zero paddings.

TABLE I
The hyperparameters of the LCN models found on the five ICBEB
experiments. The most common architectures are in bold font.

| | |
|---|---|
| ReLU-LCN | Leaky-LCN |

| Repeat | nᵣₑₚₑₐₜ | skip | bn | nᵣₑₚₑₐₜ | skip | bn |
|---|---|---|---|---|---|---|
| 1 | 5 | + | + | 7 | + | + |
| 2 | 6 | + | + | 5 | + | + |
| 3 | 4 | + | + | 5 | + | + |
| 4 | 5 | + | + | 5 | + | + |
| 5 | 5 | + | + | 5 | + | + |

5) Results: The model architecture and training characteristics of ReLU-LCN, Leaky-LCN, and the Hannun-Rajpurkar model are shown in Table II. The number of parametric layers are of the most frequently found architecture among the 5 experiments, and the speed (s/epoch) and total epochs are the average value over the five experiments. The runtime is calculated by equation (26). The identified “best” architectures were identical for ReLU-LCN and Leaky-LCN, both have only 2.3% parameters compared to the Hannun-Rajpurkar model. Both ReLU-LCN and Leaky-LCN converged at deeper architectures than Hannun-Rajpurkar model, which agrees with our hypothesis that the parsimony of LCN encourages the model to grow deeper.

$$runtime = \frac{1}{5} {\sum }_{i = 1}^{5} total epoch \times speed \qquad ( 26 )$$

Both LCN models compute each epoch aster than Hannun-Rajpurkar model, although the latter converged in fewer epochs (Table II). Both LCN models need much less average runtime than the Hannun-Rajpurkar model. The training speed not only depends on the architecture but also on the input signal length and the batch size (the longer the signal, the smaller the batch size, the slower it is to train). Thus the runtime comparison between the LCN models and the Hannun-Rajpurkar model is less dramatic than the parameter comparison. On average, Leaky-LCN needed more runtime as it tended to find deeper models than ReLU-LCN (Table I).

TABLE II
The architecture and training characteristics of ReLU-LCN, Leaky-
LCN, and the Hannun-Rajpurkar models on ICBEB. conv: convolutional
layer; BN: batch normalisation; TDS: time distributed softmax.

| | ReLU-LCN | | Leaky-LCN | | Hannun-Rajpurkar | |
|---|---|---|---|---|---|---|
| Train size | 6,427 | | 6,427 | | 6,427 | |
| Test size | 450 | | 450 | | 450 | |
| Batch size | 32 | | 32 | | 32 | |
| Parametric | 84 (41 conv, | | 84 (41 conv, | | 67 (33 conv, | |
| layers | 42 BN, 1 TDS) | | 42 BN, 1 TDS) | | 33 BN, 1 TDS) | |
| Parameters (%)* | 239,596 | (2.3) | 239,596 | (2.3) | 10,473,322 | (100) |
| Speed (s/epoch) | 36 | | 41 | | 91 | |
| Total epoch | 27 | | 30 | | 21 | |
| Runtime (s, %)* | 955 | (50.0) | 1,248 | (65.3) | 1,911 | (100) |

*% relative to the Hannun-Rajpurkar model.

Table III shows the test F₁ of the three models. We can see that Leaky-LCN has the highest mean in most cases, while ReLU-LCN is comparable to Hannun-Rajpurkar in most cases. For sub-abnormal groups and the 9-class F₁, which the Challenge used as the evaluation criteria, Leaky-LCN performed universally better than the other two models. Surprisingly, all three models performed best in the LBBB class, despite that LBBB is the second smallest class in the training set. It may be explained by the fact that LBBB has clear clinical ECG diagnosis criterion. The model performances did not seem to correlate highly with the training size: STE has the similar number of training examples as LBBB but is poorly classified. It suggests certain medical conditions are inherently difficult for CNN based architectures to classify from ECG, which agrees with the clinical knowledge that some conditions do not have definite ECG characteristics.

To compare with the performance of the winning team, we took the ReLU-LCN model found in the first experiment and performed 10-fold model averaging. Our model and obtained 0.854 9-class F1 which outperformed the winning team (F₁=0.837). We chose to average ReLU-LCN model instead of the Leaky-LCN model because there is no statistical difference between the F₁ scores of the two models, but the latter has significantly higher runtime cost.

TABLE III
Mean and standard deviation of the test F₁ on five experiments
by ReLU-LCN, Leaky-LCN, and Hannun-Rajpurkar models on ICBEB.
The highest F₁ of each category is in bold font. No
model averaging was performed.

| | Training | | | Hannun- |
|---|---|---|---|---|
| | size | ReLU-LCN | Leaky-LCN | Rajpurkar |
| N | 868 | 64.1 ± 3.8 | 64.8 ± 6.0 | 69.8 ± 4.4 |
| AF | 1,048 | 84.2 ± 3.3 | 85.4 ± 1.4 | 84.7 ± 3.7 |
| I-AVB | 654 | 84.2 ± 1.9 | 85.2 ± 3.1 | 86.0 ± 3.7 |
| LBBB | 1.57 | 89.1 ± 1.7 | 88.7 ± 2.4 | 88.0 ± 2.0 |
| RBBB | 1.645 | 76.5 ± 3.4 | 78.4 ± 4.6 | 76.0 ± 4.1 |
| PAC | 506 | 64.8 ± 12.6 | 67.5 ± 4.3 | 61.4 ± 9.7 |
| PVC | 622 | 81.4 ± 4.7 | 83.1 ± 2.7 | 80.1 ± 5.6 |
| STD | 775 | 68.1 ± 6.9 | 76.2 ± 5.1 | 78.9 ± 4.7 |
| STE | 152 | 68.1 ± 3.9 | 69.2 ± 2.8 | 58.3 ± 7.7 |
| 9-class F₁ | | 75.6 ± 3.6 | 77.6 ± 2.0 | 75.9 ± 2.9 |
| F_{AF} | | 84.2 ± 3.3 | 85.4 ± 1.4 | 84.7 ± 3.7 |
| F_{Block} | | 83.3 ± 2.1 | 84.1 ± 2.1 | 83.0 ± 2.3 |
| F_{PC} | | 72.0 ± 9.3 | 75.0 ± 3.1 | 70.7 ± 7.1 |
| F_{ST} | | 68.1 ± 4.5 | 72.5 ± 3.0 | 69.9 ± 4.0 |

Note that these results were higher than the winning team despite being trained on fewer data. The winning team by Chen et al. used 6,877 training examples, also tested on 450 test cases (exclusive from the 6,877 training cases), and padded the signals to 144 s, while ReLU-LCN was trained on 6,427 recordings, and the signals are padded to only 35 s. Although the winning team's exact architecture is unknown, their model is based on bidirectional GRU (a type of RNN), which is known to be slow to train; their input signal length is about 4 times of the input to the ReLU-LCN; and they needed to average over 130 models, while ReLU-LCN only needed to average over 10 models to obtain the above results.

### PhysioNet Dataset

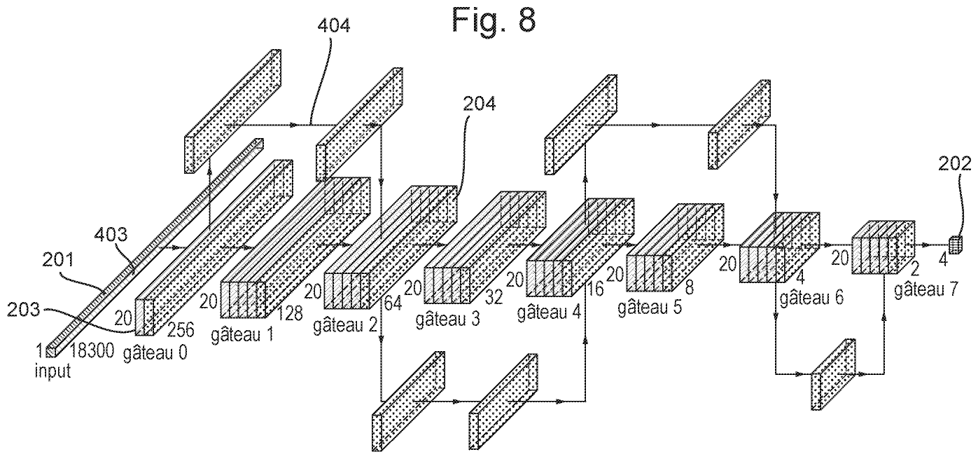

1) Train-Validation-Test Split: We randomly selected 30 samples (roughly 10% of the smallest class) from each class to build a balanced test set (n=120), and another 25 samples (roughly 9% of the smallest class) from each class to build a balanced validation set, and the rest of the dataset is the training set, as shown in FIG. 8. We repeated it five times.
2) Sample Weighting: The samples were weighted using the same procedure as described above.
3) Signal Padding: All signals were padded similarly as described above.
4) Model Generation: AutoNet identifies the “best” ReLU-LCN model and the “best” Leaky-LCN model separately in each repeat. The hyperparameter n_{f} is calculated according to equations (14) and (15) with m=8308, thus n_{f}=20. nₘₐₓₚₒₒₗ is calculated according to equation (13) with fₛ=300 Hz, τ=1 s, p=2, to be 8. It took AutoNet 53 min (3203 s) on average to identify the best ReLU-LCN model and 1 h 30 min (5413 s) to identify the best Leaky-LCN model. For ReLU-LCN, 2 out of 5 repeats converged at nᵣₑₚₑₐₜ=2 without skip connections nor batch normalisation (Table IV); 1 experiment converged at nᵣₑₚₑₐₜ=2, with only skip connections and without batch normalisation; 1 experiment converged at nᵣₑₚₑₐₜ=3, with both skip connections and batch normalisation; and the other repeat converged at nᵣₑₚₑₐₜ=4 with only skip connections and without batch normalisation. For Leaky-LCN, 4 out 5 repeats converged at nᵣₑₚₑₐₜ=4, with both skip connections and batch normalisation (FIG. 8), and the other repeat converged at nᵣₑₚₑₐₜ=5, with only skip connections and without batch normalisation.

TABLE IV
The hyperparameters of the LCN models found on the five PhysioNet
experiments The most common architectures are in bold font.

| | |
|---|---|
| ReLU-LCN | Leaky-LCN |

| Repeat | nrepeat | skip | bn | nrepeat | skip | bn |
|---|---|---|---|---|---|---|
| 1 | 3 | + | + | 4 | + | + |
| 2 | 4 | + | − | 5 | + | − |
| 3 | 2 | + | − | 4 | + | + |
| 4 | 2 | − | − | 4 | + | + |
| 5 | 2 | − | − | 4 | + | + |

FIG. 8 shows the most commonly auto-generated Leaky-LCN for PhysioNet: nᵣₑₚₑₐₜ=4, nₘₐₓₚₒₒₗ=8, c=k=20. A batch normalisation layer 403 is added after the input layer 201 and after every convolutional layer 203. Only one batch normalisation layer 203 is illustrated to declutter the figure. A skip 404 after-convolution tensor is added to every 7 subsequent after-convolution tensors.
5) Results: The model architecture and training characteristics of the three models are shown in Table V. The LCN models have no more than 2.2% of the parameters than those of the Hannun-Rajpurkar model. The same conclusions regarding runtime, total epochs, and training speed as in ICBEB hold in PhysioNet experiments, suggesting the LCNs behave consistently on different datasets.

TABLE V
The architecture and training characteristics of ReLU-LCN, Leaky-
LCN, and the Hannun-Rajpurkar model on PhysioNet. conv: convolutional
layer; BN: batch normalisation; TDS: time distributed softmax.

| | ReLU-LCN | | Leaky-LCN | | Hannun-Rajpurkar | |
|---|---|---|---|---|---|---|
| Training size | 8,308 | | 8,308 | | 8,308 | |
| Test size | 120 | | 120 | | 120 | |
| Batch size | 32 | | 32 | | 32 | |
| Parametric | 16 (15 Conv, | | 60 (29 conv, | | 67 (33 conv, | |
| layers | 1 TDS ) | | 30 BN, 1 TDS) | | 33 BN, 1 TDS) | |
| Parameters (%)* | 112,784 | (1.1) | 226,226 | (2.2) | 104,661,48 | (100) |
| Speed (s/epoch) | 20.6 | | 43.2 | | 121 | |
| Total epoch | 30 | | 28 | | 21 | |
| Runtime (s, %) | 611 | (23.6) | 1,207 | (46.6) | 2,589 | (100) |

*% relative to the Hannun-Rajpurkar model.

Table VI shows the test F₁ of the three models. We can see ReLU-LCN is better at identifying atrial fibrillation and noise while the Leaky-LCN model gave the best normal and “other rhythms” classification among the three models. Similarly, all three models are not biased towards large classes, suggesting the sample weighting mechanism is effective.

TABLE VI
The mean and standard deviation of the test F₁ in five
experiments by ReLU-LCN, Leaky-LCN, and Hannun-Rajpurkar
models on PhysioNet. The highest F1 of each category
is in bold font. No model averaging was performed.

| | Training | | | Hannun- |
|---|---|---|---|---|
| | size | ReLU-LCN | Leaky-LCN | Rajpurkar |
| AF | 708 | 88.8 ± 2.8 | 80.4 ± 2.3 | 87.9 ± 4.2 |
| Normal | 5,020 | 80.3 ± 3.6 | 86.4 ± 4.3 | 77.0 ± 12.0 |
| Other rhythms | 2,426 | 72.3 ± 7.7 | 79.5 ± 3.7 | 74.6 ± 3.8 |
| Noise | 254 | 87.9 ± 4.3 | 72.4 ± 4.6 | 74.7 ± 6.1 |
| F₁₄ | | 82.3 ± 3.1 | 83.3 ± 5.2 | 78.5 ± 3.3 |
| F₁₃ | | 80.5 ± 3.6 | 79.5 ± 1.5 | 79.8 ± 2.6 |

### CKBDataset

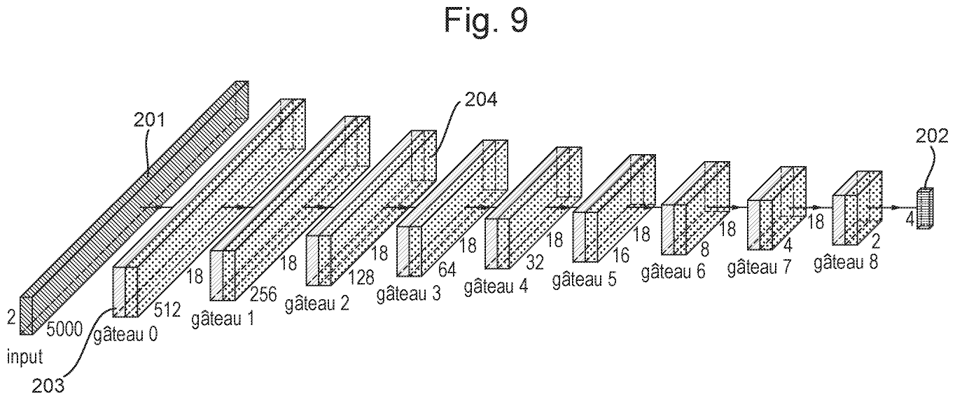

1) Train-Validation-Test Split: Due to memory constraints, we could not train on all the recordings. Therefore we constructed the largest balanced set of normal, arrhythmia, ischemia, and hypertrophy classes by randomly sampling 1,868 (the size of the smallest class) recordings from each of the four classes. The resulting set is then stratified at 8.1 10.9:1 ratio into training, validation, and test sets, respectively (FIG. 9). The sampling and split is repeated five times to generate five sets of the training, validation, and test sets for five repeats of the experiment. In each repeat, the training, validation, and test sets are shared among all models.
2) Sample Weighting: The procedure is described above.
3) Signal Padding: All signals in CKB have the same duration (10 s, 500 Hz), thus there is no need for signal padding.
4) Model Generation: The hyperparameter n_{f} is calculated according to equations (17) and (18) with m=6,056, thus n_{f}=18. nₘₐₓₚₒₒₗ is calculated according to equation (16) with fₛ=500 Hz, τ=1 s, p=2, to be 9. It took AutoNet 7 min (427 s) on average to identify the best ReLU-LCN model and 11 min (693 s) to identify the best Leaky-LCN model. For ReLU-LCN, all five repeats converged at nᵣₑₚₑₐₜ=1 without skip connections nor batch normalisation (FIG. 9); for Leaky-LCN, three out of five repeats converged at nᵣₑₚₑₐₜ=1, without skip connections nor batch normalisation, while the other 2 repeats converged at nᵣₑₚₑₐₜ=2, with only skip connections and without batch normalisation (Table VII).

TABLE VII
The hyperparameters of the LCN models found on the five CKB
experiments. The most common architectures are in bold font.

| | ReLU-LCN | | | Leaky-LCN | | |
|---|---|---|---|---|---|---|
| Repeat | nᵣₑₚₑₐₜ | skip | bn | nᵣₑₚₑₐₜ | skip | bn |
| 1 | 1 | − | − | 2 | + | − |
| 2 | 1 | − | − | 1 | − | − |
| 3 | 1 | − | − | 2 | + | − |
| 4 | 1 | − | − | 1 | − | − |
| 5 | 1 | − | − | 1 | − | − |

FIG. 9 illustrates the auto-generated network for CKB: nᵣₑₚₑₐₜ=3, nₘₐₓₚₒₒₗ=9, n_{f}=k=18. A single convolutional (+activation) layer 203 is included between each pair of pooling layers 204. No batch normalisation nor skip connection was needed. The output 202 is a 4-unit time distributed softmax layer.
5) Results: The model architecture and training characteristics of the three models are shown in the Table VIII. Both LCN models converged at nine convolutional layers without the need for batch normalisation, with only 0.5% parameters and needed five times less runtime than the Hannun-Rajpurkar model.

Table XI shows the test set classification F₁ of the three models. LCN models outperformed the Hannun-Rajpurkar model universally, with 8-16% improvement on performance depending on the category and model. ReLU-LCN performed best in most categories, except ischemia, but the difference with Leaky-LCN and ReLU-LCN is insignificant In this dataset, both training and test sets are balanced, so the difference given by the same model comes solely from the nature of the medical condition. Arrhythmia and ischemia were more difficult for all three models while hypertrophy was the easiest. This agrees with the result in ICBEB where LBBB was the best classified.

TABLE VIII
The architecture and training characteristics of ReLU-LCN, Leaky-
LCN, and the Hannun-Rajpurkar model on CKB. conv: convolutional
layer; BN: batch normalisation; TDS: time distributed softmax.

| | ReLU-LCN | | Leaky-LCN | | Hannun-Rajpurkar | |
|---|---|---|---|---|---|---|
| Training size | 6,728 | | 6,728 | | 6,728 | |
| Test size | 744 | | 744 | | 744 | |
| Batch size | 32 | | 32 | | 32 | |
| Parametric | 10 (9 conv, | | 10 (9 conv, | | 67 (33 conv. | |
| layers | 1 TDS) | | 1 TDS) | | 33BN, 1 TDS) | |
| Parameters (%)* | 50,782 | (0.5) | 50,7872 | (0.5) | 10,471,780 | (100) |
| Speed (s/epoch) | 4 | | 5 | | 34 | |
| Total epoch | 24 | | 20 | | 13 | |
| Runtime (s, %)* | 95 | (21.5) | 97 | (22.0) | 442 | (100) |

*% relative to the Hannun-Rajpurkar model.

TABLE XI
Mean and standard deviation of the F₁ on five experiments
by ReLU-LCN, Leaky-LCN, and Hannun-Rajpurkar models on CKB.
The highest F₁ of each category is in bold font.
No model averaging was performed.

| | Training | | | Hannun- |
|---|---|---|---|---|
| | size | ReLU-LCN | Leaky-LCN | Rajpurkar |
| Arrhythmia | 1,681 | 74.0 ± 1.4 | 71.7 ± 3.7 | 63.7 ± 10.1 |
| Hypertrophy | 1,681 | 85.2 ± 1.5 | 82.5 ± 1.0 | 75.2 ± 16.8 |
| Ischemia | 1,681 | 72.4 ± 2.6 | 73.2 ± 2.0 | 66.9 ± 2.2 |
| Normal | 1,681 | 77.2 ± 2.9 | 75.6 ± 2.7 | 69.5 ± 3.3 |
| 4-class F₁ | | 77.2 ± 1.6 | 75.8 ± 1.9 | 68.9 ± 4.6 |

This is a classic case that a large model, even if well-regularised, may not outperform a smaller model. In fact, as demonstrated in all three datasets, the smaller but carefully designed smaller network can perform from slightly better to markedly better than a larger network. Moreover, we have demonstrated that the hyperparameters of such “careful” design of networks can indeed be mathematically derived.

### Statistical Analysis

To test the applicability of a paired t-test on the F₁ of 15 experiments (Table X), we performed Shapiro-Wilk test for normality [17] on the differences between the F₁ scores obtained by the Hannun-Rajpurkar model and the ReLU-LCN model on 15 experiments (5 repeats on each of the three datasets), and found p-value=0.158>0.05. Similarly, we tested the normality of the differences between Leaky-LCN and Hannun-Rajpurkar and found p-value=0.832>0.05. Both passed the normality test (the null hypothesis of Shapiro-Wilk test of normality is that the samples come from a Gaussian distribution, thus p-value>the chosen significance level (α=0.05) fails to reject the null hypothesis, thus passing the Shapiro-Wilk test), meaning both differences do not deviate significantly from a Gaussian distribution thus appropriate for two-sided paired t-test. (As long as the sample difference does not deviate significantly from a Gaussian, it is appropriate to use paired t-tests [10]). We then did pair-wise two-tail paired t-test on the F₁ scores of the three models, and found p-value=0.023<0.05 between ReLU-LCN and Hannun-Rajpurkar, and p-value=0.012<0.05 between Leaky-LCN and Hannun-Rajpurkar, and p-value=0.667>0.05 between ReLU-LCN and Leaky-LCN. We conclude that there is a significant difference between ReLU-LCN and Hannun-Rajpurkar models, and between Leaky-ReLU and Hannun-Rajpurkar models, but no significant difference in F₁ scores were found between ReLU-LCN and Leaky-LCN. However, we cannot conclude from the above results that there are significant differences among the three models as that would require repeated measurement analysis of variance (ANOVA), the assumption of which is that samples, i.e. the 15 F₁ scores, come from a single Gaussian distribution for each model. However, the 15 F1 scores of each model failed the Shapiro-Wilk test for normality, thus not suitable for ANOVA.

### Performance-to-Computational Cost (PC) Ratio

We propose an intuitive metric to evaluate the computation efficiency of deep learning models called the Performance-to-Computational Cost (PC) ratio, to help with the decision making as to which model to try and how to improve performance from a study design perspective. The PC ratio is defined below

$$PC ratio = K \times \frac{{( performance metric )}^{p}}{{( computational cost )}^{q}} \qquad ( 27 )$$

where K is a scaling constant to scale the PC ratio to a convenient range. The higher the PC ratio, the better. The performance metric and the computational cost can be anything appropriate for the practitioner as long as it is consistent across all models and datasets. p

TABLE X
F₁ of 15 experiments using the three models. In each experiment,
the training and test sets are shared among all models. In PhysioNet,
the shown results are 4-class average F₁. The highest F₁ of
each experiment is shown in bold font.

| | | | | Hannun- |
|---|---|---|---|---|
| Dataset | Experiment | ReLU-LCN | Leaky-LCN | Rajpurkar |
| ICBEB | 1 | 81.8 | 81.5 | 77.5 |
| | 2 | 70.7 | 76.8 | 75.9 |
| | 3 | 76.8 | 75.6 | 79.6 |
| | 4 | 74.5 | 77.1 | 70.8 |
| | 5 | 74.3 | 77.0 | 75.7 |
| PhysioNet | 1 | 82.5 | 78.5 | 80.9 |
| | 2 | 87.4 | 80.1 | 73.7 |
| | 3 | 77.8 | 81.5 | 76.1 |
| | 4 | 82.4 | 84.3 | 82.9 |
| | 5 | 81.5 | 77.7 | 79.0 |
| CKB | 1 | 77.2 | 78.0 | 73.2 |
| | 2 | 76.4 | 75.1 | 61.7 |
| | 3 | 74.7 | 77.6 | 72.1 |
| | 4 | 78.7 | 75.5 | 65.3 |
| | 5 | 78.9 | 72.7 | 72.0 |

and q are constants reflecting the practitioners' emphasis on performance or computational cost. For example, here, we use p=q=1, representing an equal preference for the performance and the computational cost. Practitioners more concerned with the performance may use p=2, q=1. Using runtime cost(s) as the metric for computational cost, and F₁ as the performance metric, and K=10,000, we calculate the value for ReLU-LCN, Leaky-LCN, and Hannun-Rajpurkar model as in Table XI.

The PC ratio can compare not only different models on the same dataset but also compare different datasets using the same model. Take ReLU-LCN as an example, we can see that the PC ratios of CKB are much higher than the other two datasets, suggesting CKB is relatively easy to achieve good performance with low computational cost, perhaps due to high signal quality and a large number of training examples per class. However, in Table IX the actual F₁ in CKB is no higher than those of the other two datasets (Tables III and VI), suggesting improving upon CKB performance from the model perspective is difficult given the current dataset perhaps due to the short signal duration (10 s) compared to ICBEB (35 s) and PhysioNet (61 s). This gives us insights as to which direction to pursue if we

TABLE XI
The PC ratio, calculated as runtime(s) F₁ ×
10000. The higher the value is, the better. The
highest value of each experiment is in bold font.

| | | | | Hannun- |
|---|---|---|---|---|
| Dataset | Experiment | ReLU-LCN | Leaky-LCN | Rajpurkar |
| ICBEB | 1 | 7.1 | 3.8 | 5.0 |
| | 2 | 7.7 | 7.4 | 4.0 |
| | 3 | 8.3 | 9.7 | 4.2 |
| | 4 | 8.2 | 6.3 | 3.9 |
| | 5 | 8.6 | 6.2 | 3.5 |
| PhysioNet | 1 | 10.6 | 8.4 | 3.6 |
| | 2 | 9.3 | 5.5 | 3.7 |
| | 3 | 17.9 | 6.2 | 3.7 |
| | 4 | 19.8 | 6.4 | 3.5 |
| | 5 | 16.3 | 6.6 | 1.9 |
| CKB | 1 | 96.5 | 64.2 | 17.6 |
| | 2 | 76.4 | 70.1 | 18.4 |
| | 3 | 69.2 | 50.1 | 16.3 |
| | 4 | 85.5 | 90.7 | 20.2 |
| | 5 | 82.2 | 105.9 | 14.3 |

want to improve performance further: to improve the model, or to collect more data from the same study participants, or to recruit more study participants. A high PC ratio, such as in CKB, may suggest the number of training examples is abundant, while a low PC ratio, such as in ICBEB, may suggest the curse of dimensionality, or in other words, the number of training examples per class is insufficient to train a model that can take advantage of the high dimensional feature vector of each training example.

### DISCUSSION

Each dataset has unique challenges: ICBEB has the most numerous classes and least number of training examples per class; PhysioNet has the highest noise ratio, and has only single lead; CKB has the shortest signal duration. Comparing the test F1 across three datasets (Table X), it is encouraging to see that the lowest performance was in fact from CKB, as it implies that the bottleneck of performance lies with the amount of information contained in each training example. This suggests that LCN can indeed make the most out of the training set. It is also encouraging to see that LCN can perform well even if there are few training examples per class, which is often the limiting factor for deep learning. Also, the simple sample weighting method effectively addressed the class skewness, and the LCN models have almost no bias towards the large classes.

Table X shows that given the same experiment, it is almost always one of the LCN models that yielded the best performance. Although Hannun-Rajpurkar model seemed to be the least well-performing model in this chapter, we shall not forget that it has been proven to exceed average human cardiologists on 12 rhythm classes of 91,232 recordings from 53,549 participants [5]. LCN models outperformed the Hannun-Rajpurkar model slightly in ICBEB and PhysioNet, and markedly in CKB. The results suggest the model complexity of the Hannun-Rajpurkar model may be appropriate for ICBEB and PhysioNet but too high for CKB, which leads us to hypothesise that the model complexity of Auto-generated LCNs may be very close to the optimal model complexity given the dataset, and their test loss is close to the Bayesian loss. From this perspective, LCN may be used to estimate the real complexity of the problem. We have proposed the PC ratio as a simple measure of computational efficiency and we can see that ReLU-LCN has much higher PC ratio than the other two models. Thus we recommend ReLU-LCN. Also, the PC ratio of each dataset may be a measure of the difficulty of the classification task.

Although the final loss is not guaranteed to be convex with respect to the hidden layer weights if the network is allowed to have negative hidden activations, such as in Leaky-LCN, the LCN hidden layers are effectively over-determined systems of monotonic equations. Over-determined systems of monotonic equations have a unique solution that minimises the Euclidean distance, which is equivalent to minimising the mean squared error (MSE), which is not only convex but quadratic. Theoretically, we should use a loss which has MSE terms from each layer. In this study, we used conventional cross-entropy loss as an approximation, and it has been proven to work very well. Future work will include designing experiments to study the properties of the loss surface of LCN and experiment with alternative loss functions.

In this study, we used Adam [11] with all default hyperparameters as the optimiser, without even tuning the learning rate. Our principle is to use as many default hyperparameters as possible, including the learning rate, of a robust optimisation algorithm, such as Adam, and innovate in model architectures so that tuning optimisation hyperparameters is unnecessary.

One of the major contributions of LCN is a novel paradigm to determine the hyperparameters of CNN. Central to the LCN theorem is the choice of f and k. In the version of LCN discussed above, the kernel size k is set to be equal to n_{f}. Theoretically, k should be independently optimised to maximise the total number of parameters in each layer, subject to n_{f} (n_{f}k+1)≤m. However, for long single-lead signals, such as those in PhysioNet, k would end up being unreasonably large (for example k>300). Thus we kept k to be the same as n_{f}. This also implicitly expresses our view that the parameters in the kernels and the parameters in the channel dimension are not fundamentally different. The resulting LCN typically has no more than 2% of the parameters compared to the state of the art model, which is very encouraging as this means at least O(n_{θ}) saving in memory and computational complexity. LCN may also make second-order algorithms feasible, as many second-order methods need O(n_{θ}²) (conjugate gradient descent, BFGS) or O(n_{θ}³) (Newton method) complexity. If we optimise the parameters layer-by-layer, the computational complexity can be further reduced to be less than O(m²), where m is the number of training examples. The hypothesised Layer-Wise quadratic property suggests the second-order methods such as Newton's method may be very applicable. Future work include designing experiments to study the behaviour of convex optimisation in LCN networks. The 50-200 times fewer parameters may enable the algorithm to run on devices where it is otherwise impossible to run deep learning models. While developing the AutoNet algorithm, we found the following techniques very helpful in boosting the performance: (i) Handle class imbalance by weighting the training samples by the inverse of the class ratio in the training set. The key is to have a balanced validation set for model check-pointing, even if the final test set is not balanced. (ii) Time-distributed softmax output for periodic time series signals. (iii) Model averaging.

### REFERENCES

[1] Z. Chen, J. Chen, R. Collins, Y. Guo, R. Peto, F. Wu, and L. Li. China kadoorie biobank of 0.5 million people: survey methods, baseline characteristics and long-term follow-up. International Journal of Epidemiology, 40(6):1652-1666, 2011.
[2] G. D. Clifford, C. Liu, B. Moody, L.-w. H. Lehman, I. Silva, Q. Li, A. Johnson, and R. G. Mark. Af classification from a short single lead ecg recording: The physionet computing in cardiology challenge 2017. Proceedings of Computing in Cardiology, 44:1, 2017.
[3] G. Cybenko. Approximation by superpositions of a sigmoidal function. Mathematics of Control, Signals and Systems, 2(4):303-314, 1989.
[4] B. Hanin. Which neural net architectures give rise to exploding and vanishing gradients? In NeurIPS, pages 582-591, 2018.
[5] A. Y. Hannun, P. Rajpurkar, M. Haghpanahi, G. H. Tison, C. Bourn, M. P. Turakhia, and A. Y. Ng. Cardiologist-level arrhythmia detection and classification in ambulatory electrocardiograms using a deep neural network. Nature Medicine, 25(1):65, 2019.
[6] K. He, X. Zhang, S. Ren, and J. Sun. Deep residual learning for image recognition. In CVPR, pages 770-778, 2016.
[7] K. Hornik, M. Stinchcombe, and H. White. Multilayer feedforward networks are universal approximators. Neural Networks, 2(5):359-366, 1989.
[8] K. Hornik, M. Stinchcombe, and H. White. Universal approximation of an unknown mapping and its derivatives using multilayer feedforward networks. Neural Networks, 3(5):551-560, 1990.
[9] S. Ioffe and C. Szegedy. Batch normalization: Accelerating deep network training by reducing internal covariate shift. arXiv preprint arXiv:1502.03167, 2015.
[10] E. T. Jaynes. Probability theory: The logic of science. Cambridge University Press, Cambridge, 2003.
[11] D. P. Kingma and J. Ba. Adam: A method for stochastic optimization. arXiv preprint arXiv:1412.6980, 2014.
[12] A. Krizhevsky, I. Sutskever, and G. E. Hinton. Imagenet classification with deep convolutional neural networks. In NeurIPS, pages 1097-1105, 2012.
[13] Y. LeCun, L. Bottou, Y. Bengio, P. Haffner, et al. Gradient-based learning applied to document recognition. Proceedings of the IEEE, 86(11):2278-2324, 1998.
[14] M. Leshno, V. Y. Lin, A. Pinkus, and S. Schocken. Multilayer feedforward networks with a nonpolynomial activation function can approximate any function. Neural Networks, 6(6):861-867, 1993.
[15] P. Rajpurkar, A. Y. Hannun, M. Haghpanahi, C. Bourn, and A. Y. Ng. Cardiologist-level arrhythmia detection with convolutional neural networks. arXiv preprint arXiv:1707.01836, 2017.
[16] O. Russakovsky, J. Deng, H. Su, J. Krause, S. Satheesh, S. Ma, Z. Huang, A. Karpathy, A. Khosla, M. Bernstein, et al. ImageNet large scale visual recognition challenge. International Journal of Computer Vision, pages 211-252, 2015.
[17] S. S. Shapiro and M. B. Wilk. An analysis of variance test for normality (complete samples). Biometrika, 52(3/4):591-611, 1965.
[18] K. Simonyan and A. Zisserman. Very deep convolutional networks for large-scale image recognition. In ICLR, 2015.

1. A computer-implemented method for generating a neural network comprising:
receiving input data;
determining values of a plurality of hyperparameters based on one or more properties of the input data;
generating, based on the values of the hyperparameters, a neural network comprising a plurality of layers;
training the neural network using the input data and, at least if a first predetermined condition is not met, updating the values of one or more of the hyperparameters;
repeating the steps of generating a neural network, and training the neural network until the first predetermined condition is met;
selecting one of the trained neural networks; and
outputting the selected neural network.

2. The method of claim 1, wherein the plurality of layers comprises one or more pooling layers and one or more convolutional layers between each pooling layer, the plurality of hyperparameters comprising the number of pooling layers and the number of convolutional layers between each pooling layer.

3. The method of claim 2, wherein the pooling layers are maxpooling layers.

4. The method of claim 2, wherein the input data is periodic time series data, and in the step of determining values of the plurality of hyperparameters, the number of pooling layers is determined based on a number of samples in the time series data per period of the time series data.

5. The method of claim 4, wherein the number of pooling layers is determined according to:
nₘₐₓₚₒₒₗ=┌logₚ(fₛτ)┐
where nₘₐₓₚₒₒₗ is the number of pooling layers, p is a predetermined parameter quantifying a reduction in dimensionality by each pooling layer, τ is a predetermined estimate of the period of the time series data, and fₛ is a sampling frequency of the time series data.

6. The method of claim 2, wherein the input data is non-periodic time series data, and in the step of determining values of the plurality of hyperparameters, the number of pooling layers is determined based on a number of samples in the time series data.

7. The method of claim 6, wherein the number of pooling layers is determined according to:
nₘₐₓₚₒₒₗ=┌logₚ(D)┐
where nₘₐₓₚₒₒₗ is the number of pooling layers, p is a predetermined parameter quantifying a reduction in dimensionality by each pooling layer, and D is a number of samples of the time series data.

8. The method of claim 2, wherein the plurality of layers further comprises an activation layer following each convolutional layer.

9. The method of claim 8, wherein the activation layer comprises a rectified linear unit or a leaky rectified linear unit.

10. The method of claim 2, wherein updating the values of one or more of the hyperparameters comprises increasing the number of convolutional layers between each pooling layer.

11. The method of claim 1, wherein the input data is labelled input data and the neural network is trained using supervised learning.

12. The method of claim 11, wherein:
the plurality of layers comprises one or more pooling layers and one or more convolutional layers between each pooling layer, the plurality of hyperparameters comprising the number of pooling layers and the number of convolutional layers between each pooling layer;
each convolutional layer has an associated plurality of parameters, and training the neural network comprises:
choosing values of the parameters of the convolutional layers based on the values of the hyperparameters and the previous values of the parameters of the convolutional layers;
calculating a training value of a loss function using an output of the neural network; and
repeating the steps of choosing values of the parameters and calculating the value of the loss function until a change in the training value of the loss function over two or more consecutive steps of calculating the training value of the loss function is below a predetermined threshold.

13. The method of claim 12, wherein the training value of the loss function comprises a training loss calculated by evaluating the loss function on the output of the neural network applied to the input data.

14. The method of claim 1, wherein the first predetermined condition is met when a validation value of a loss function optionally wherein a validation loss calculated by evaluating the loss function on the output of the neural network applied to a validation data set, of the neural network following the step of training the neural network is not lower than the validation value of the loss function of the neural network following the training of the previous neural network.

15. The method of claim 1, wherein the method further comprises, after the first predetermined condition is met:
generating, based on the values of the hyperparameters, a neural network comprising one or more skip connections between non-consecutive layers of the neural network;
training the neural network comprising one or more skip connections using the input data and, at least if a second predetermined condition is not met, updating the values of one or more of the hyperparameters; and
repeating the steps of generating a neural network comprising one or more skip connections and training the neural network comprising one or more skip connections until the second predetermined condition is met.

16. The method of claim 15, wherein the second predetermined condition is met when a validation value of a loss function, optionally wherein a validation loss calculated by evaluating the loss function on the output of the neural network applied to a validation data set, of the neural network comprising one or more skip connections following the step of training the neural network comprising one or more skip connections is not lower than the validation value of the loss function of the neural network comprising one or more skip connections following the training of the previous neural network comprising one or more skip connections.

17. The method of claim 15, wherein the method further comprises, after the second predetermined condition is met:
generating, based on the values of the hyperparameters, a neural network comprising one or more batch normalisation layers;
training the neural network comprising one or more batch normalisation layers using the input data and, at least if a third predetermined condition is not met, updating the values of one or more of the hyperparameters; and
repeating the steps of generating a neural network comprising one or more batch normalisation layers and training the neural network comprising one or more batch normalisation layers until the third predetermined condition is met.

18. The method of claim 17, wherein the plurality of layers comprises a plurality of convolutional layers and an activation layer following each convolutional layer, and the neural network comprising one or more batch normalisation layers comprises a batch normalisation layer following each activation layer.

19. The method of claim 17, wherein the third predetermined condition is met when a validation value of a loss function, optionally wherein a validation loss calculated by evaluating the loss function on the output of the neural network applied to a validation data set, of the neural network comprising one or more batch normalisation layers following the step of training the neural network comprising one or more batch normalisation layers is not lower than the validation value of the loss function of the neural network comprising one or more batch normalisation layers following the previous step of training the neural network comprising one or more batch normalisation layers.

20. (canceled)

21. The method of claim 1, wherein the input data comprises time series data.

22. The method of claim 21, wherein the time series data is cyclic physiological data.

23. The method of claim 22, wherein the time series data is electrocardiogram data.

24. The method of claim 1, wherein selecting one of the trained neural networks comprises selecting the trained neural network having a lowest validation value of a loss function.

25. The method of claim 1, wherein selecting one of the trained neural network comprises:
training the neural network having a lowest validation value of a loss function a plurality of times to obtain a corresponding plurality of trained instances of the neural network having the lowest validation value of the loss function; and
providing as the selected neural network an average ensemble of the trained instances.

26. The method of claim 24, wherein the validation value of the loss function comprises a validation loss calculated by evaluating the loss function on the output of the trained neural network applied to a validation data set.

27. The method of claim 1, wherein outputting the selected neural network comprises outputting the values of the hyperparameters used in generating the selected neural network.

28. The method of claim 1, wherein the plurality of layers comprises one or more convolutional layers, each convolutional layer having an associated plurality of parameters, and outputting the selected neural network comprises outputting the values of the parameters of the convolutional layers.

29. The method of claim 1, wherein the neural network further comprises a classification layer.

30. The method of claim 29, wherein the input data is physiological data, and the classification layer is configured to classify the input data into one of a plurality of clinical categories.

31. A method of classifying physiological data from a patient, the method comprising:
receiving the physiological data;
generating a neural network according to the method of claim 29, wherein the input data is the physiological data; and
using the neural network to classify the physiological data.

32. A method of classifying a patient into a clinical category, the method comprising:
receiving the physiological data;
generating a neural network according to the method of claim 30;
using the neural network to classify the physiological data; and
classifying the patient into one of a plurality of clinical categories based on the classification of the physiological data from the classification layer of the neural network.

33. A computer program comprising instructions which, when the program is executed by a computer, cause the computer to carry out the method of claim 1.

34. A computer-readable medium comprising instructions which, when executed by a computer, cause the computer to carry out the method of claim 1.

35. An apparatus for generating a machine-learning network comprising:
a receiving unit configured to receive input data comprising time series data, and a processing unit configured to:
determine values of a plurality of hyperparameters based on one or more properties of the input data;
generate, based on the values of the hyperparameters, a convolutional neural network comprising a plurality of layers;
train the neural network using the input data and, at least if a first predetermined condition is not met, updating the values of one or more of the hyperparameters;
repeat the steps of generating a neural network, and training the neural network until the first predetermined condition is met;
select one of the trained neural networks; and
output the selected neural network.
